(19) United States
(12) Patent Application Publication
(10) Pub. No.: US 2020/0405245 A1
(43) Pub. Date: Dec. 31, 2020

(54) BIOLOGICAL INFORMATION DISPLAY DEVICE, BIOLOGICAL INFORMATION DISPLAY METHOD AND PROGRAM

(71) Applicant: PANASONIC INTELLECTUAL PROPERTY MANAGEMENT CO., LTD., Osaka (JP)

(72) Inventors: Tadanori TEZUKA, Fukuoka (JP); Tsuyoshi NAKAMURA, Fukuoka (JP)

(73) Assignee: PANASONIC INTELLECTUAL PROPERTY MANAGEMENT CO., LTD., Osaka (JP)

(21) Appl. No.: 16/968,489

(22) PCT Filed: Feb. 8, 2019
(86) PCT No.: PCT/JP2019/004685

§ 371 Date: Aug. 7, 2020

(30) Foreign Application Priority Data
Feb. 13, 2018 (JP) 2018-023436

(51) Int. Cl. A61B 5/00; A61B 5/0205

(52) U.S. Cl. CPC A61B 5/742; A61B 5/0205; A61B 5/441; A61B 5/0077

(57) ABSTRACT

A biological information display device has a camera, a monitor, a CPU as a processing unit, and a DSP, and the camera and the monitor are positioned on the same surface in a housing. The camera acquires, from captured images, video data of a detected region comprising a section of skin on the face of a subject being monitored, and the processing unit acquires, from the video data, biological information comprising information about the pulse wave of a person, and generates an information display screen, which include a respiration assistant that assists the respiration of the subject being monitored. In the information display screen, at least one portion of the breathing assistant is positioned on a straight line that passes through the camera, and the straight line that passes through the camera perpendicularly crosses the outer peripheral edge of the monitor closest to the camera.

### TECHNICAL FIELD

The present invention relates to a vital information display apparatus, a vital information display method, and a program for detecting and displaying human vital information.

### BACKGROUND ART

Vital sensing technologies for detecting human vital information are expected to be applied to a wide variety of fields including, for example, not only the fields of home medical care and health care but also detection of sleepiness during a drive, acquisition of a psychological state of a user who is playing a game, and detection of an abnormal person by a surveillance system. Various studies have been made of methods for evaluating psychological stress by analyzing a state of a human autonomic nervous system from vital information such as a heart rate, a breathing rate, and a pulse wave by applying vital sensing technologies to them. Furthermore, instruments have been proposed that perform what is called heart rate variability biofeedback which increases the activity of the parasympathetic nerves of the autonomic nervous system and thereby reduces stress by providing a user with vital information using such a psychological stress evaluation method (refer to PTL 1, for example).

### CITATION LIST

### Patent Literature

[PTL1] JP-T-2008-536648

### SUMMARY OF INVENTION

### Technical Problem

Conventional instruments that perform heart rate variability biofeedback are configured so as to be used in such a manner that a sensor unit for detecting vital information is brought into contact with a human body. Thus, when such an instrument is used, a user himself or herself is required to do a cumbersome act of attaching the sensor unit to him or her or keeping, for example, his or her finger in contact with the sensor unit. As such, conventional instruments have a problem that they can be used only in limited environments and are desired to be increased in usability.

The concept of the present disclosure has been conceived in view of the above-described circumstances in the art, and an object of the present invention is to provide a vital information display apparatus, a vital information display method, and a program capable of increasing the usability when heart rate variability biofeedback is performed.

### Solution to Problem

The present disclosure provides a vital information display apparatus comprising a body; an imaging unit which images a face of an observation target person and acquires, from an image taken, video data of a detection area including a skin portion of the face of the observation target person; a processing unit which acquires vital information including information of a human pulse wave from the video data of the detection area and generates an information display picture to be used for performing heart rate variability biofeedback using the vital information; and a display unit which is installed on the same surface in the body as the imaging unit and displays the information display picture, wherein the processing unit generates, for the heart rate variability biofeedback, an information display picture including a breathing assistant that assists breathing of the observation target person; and in the information display picture, at least part of the breathing assistant is disposed on a straight line passing through the imaging unit and the straight line passing through the imaging unit crosses a side, closest to the imaging unit, of an outer circumference of the display unit perpendicularly.

The disclosure also provides a vital information display method employed in a vital information display apparatus which comprises a body, an imaging unit which images a face of an observation target person, and a display unit which displays vital information acquired from an image taken and in which the display unit is disposed on the same surface as the imaging unit in the body, comprising the steps of imaging the face of the observation target person by the imaging unit and acquiring, from an image taken, video data of a detection area including a skin portion of the face of the observation target person; acquiring vital information including information of a human pulse wave from the video data of the detection area and generates an information display picture to be used for performing heart rate variability biofeedback using the vital information; in generating an information display picture, generating, for the heart rate variability biofeedback, an information display picture including a breathing assistant that assists breathing of the observation target person; and displaying the information display picture on the display unit, wherein in the information display picture, at least part of the breathing assistant is disposed on a straight line passing through the imaging unit and the straight line passing through the imaging unit crosses a side, closest to the imaging unit, of an outer circumference of the display unit perpendicularly.

The disclosure further provides a program for causing a computer to execute steps of a vital information display method in a vital information display apparatus which comprises a body, an imaging unit which images a face of an observation target person, and a display unit which displays vital information acquired from an image taken and in which the display unit is disposed on the same surface as the imaging unit in the body, the steps comprising: imaging the face of the observation target person by the imaging unit and acquiring, from an image taken, video data of a detection area including a skin portion of the face of the observation target person; acquiring vital information including information of a human pulse wave from the video data of the detection area and generates an information display picture to be used for performing heart rate variability biofeedback using the vital information; in generating an information display picture, generating, for the heart rate variability biofeedback, an information display picture including a breathing assistant that assists breathing of the observation target person; and displaying the information display picture on the display unit, wherein in the information display picture, at least part of the breathing assistant is disposed on a straight line passing through the imaging unit and the straight line passing through the imaging unit crosses a side, closest to the imaging unit, of an outer circumference of the display unit perpendicularly.

### Advantageous Effects of Invention

The disclosure makes it possible to increase the usability when heart rate variability biofeedback is performed.

### BRIEF DESCRIPTION OF DRAWINGS

FIG. 1 is a block diagram showing an example configuration of a vital information display apparatus according to an embodiment.

FIG. 2A is a schematic diagram showing an example relationship between contraction of a human heart and the amount of absorption of light by blood vessels.

FIG. 2B is a graph showing an example time-series variation of light intensity.

FIG. 3 is a graph showing an example of how the absorbance of hemoglobin varies depending on the wavelength of light.

FIG. 4 is a diagram showing example of signals of vital information extracted from a video signal of an image taken.

FIG. 5A is for description of an example (Display example-1) of an information display picture of the vital information display apparatus according to the embodiment.

FIG. 5B is for description of an example of the information display picture of Comparative Example.

FIG. 6 is a flowchart showing a processing procedure to be followed in a heart rate variability biofeedback operation performed in the embodiment.

FIG. 7 is a diagram for description of RRI output timing.

FIG. 8A is a diagram showing Display example-2 of the information display picture employed in the embodiment.

FIG. 8B is a diagram showing Display example-3 of the information display picture employed in the embodiment.

FIG. 9 is a diagram showing Display example-4 of the information display picture employed in the embodiment.

FIG. 10A is a diagram showing Display example-1 of a breathing pacer displayed in the information display picture employed in the embodiment.

FIG. 10B is a diagram showing Display example-2 of the breathing pacer displayed in the information display picture employed in the embodiment.

FIG. 10C is a diagram showing Display example-3 of the breathing pacer displayed in the information display picture employed in the embodiment.

FIG. 10D is a diagram showing Display example-4 of the breathing pacer displayed in the information display picture employed in the embodiment.

FIG. 10E is a diagram showing Display example-5 of the breathing pacer displayed in the information display picture employed in the embodiment.

FIG. 10F is a diagram showing Display example-6 of the breathing pacer displayed in the information display picture employed in the embodiment.

FIG. 11 is a flowchart showing a processing procedure to be followed when the orientation of the information display picture for heart rate variability biofeedback employed in the embodiment has been changed.

FIG. 12A is a diagram for description of an example (Display example-5) of the information display picture of the vital information display apparatus according to the embodiment.

FIG. 12B is a diagram for description of an example of the information display picture of Comparative Example.

FIG. 13A is a diagram showing Arrangement example-1 of GUIs in the information display picture employed in the embodiment.

FIG. 13B is another diagram showing Arrangement example-1 of GUIs in the information display picture employed in the embodiment.

FIG. 14A is a diagram showing Arrangement example-2 of GUIs in the information display picture employed in the embodiment.

FIG. 14B is another diagram showing Arrangement example-2 of GUIs in the information display picture employed in the embodiment.

FIG. 15A is a diagram showing Arrangement example-3 of GUIs in the information display picture employed in the embodiment.

FIG. 15B is another diagram showing Arrangement example-3 of GUIs in the information display picture employed in the embodiment.

FIG. 16A is a diagram showing Arrangement example-4 of GUIs in the information display picture employed in the embodiment.

FIG. 16B is another diagram showing Arrangement example-4 of GUIs in the information display picture employed in the embodiment.

### DESCRIPTION OF EMBODIMENTS

An embodiment as a specific disclosure of a vital information display apparatus, a vital information display method, and a program according to the present disclosure will be described in detail by referring to the drawings when necessary. However, unnecessarily detailed descriptions may be avoided. For example, detailed descriptions of already well-known items and duplicated descriptions of constituent elements having substantially the same ones already described may be omitted. This is to prevent the following description from becoming unnecessarily redundant and thereby facilitate understanding of those skilled in the art. The following description and the accompanying drawings are provided to allow those skilled in the art to understand the disclosure thoroughly and are not intended to restrict the subject matter set forth in the claims.

### (Background Leading to Embodiment of Disclosure)

The embodiment is directed to an example configuration, for detecting vital information without making contact using an image taken and displaying it, of a vital information display apparatus for displaying vital information for heart rate variability biofeedback.

A conventional instrument is configured in such a manner that a sensor unit for detecting vital information is brought into contact with a human body in performing heart rate variability biofeedback and requires a user himself or herself to do a cumbersome act of attaching the sensor unit to him or her or keeping, for example, his or her finger in contact with the sensor unit when the instrument is used. In view of this, a case of detecting vital information without making contact in heart rate variability biofeedback is assumed.

A technique for estimating vital information such as a heart rate and a pulse wave from an image taken by a camera has been proposed as a method for detecting vital information without making contact. To detect vital information wave from an image taken, a face portion of an observation target person who is a user is detected using a known face detection technique, the face portion as a skin portion is set as a prescribed detection area of the image, and vital information is detected from the prescribed detection area. In this case, vital information can be detected with high accuracy by taking the following two points into consideration:

(1) The user face is set so as to be as close to be square with the camera as possible. With this measure, face detection processing can be performed normally. Furthermore, since the area of the face portion (i.e., skin portion) can be made as large as possible, a heart rate measurement can be performed accurately.

(2) The user face is set as still as possible. This makes it possible to perform a heart rate measurement stably and hence to detect vital information with higher accuracy.

In view of the above, in the following embodiment, an example configuration that makes it possible to increase the usability by detecting vital information without making contact from an image taken by imaging a user face and performing heart rate variability biofeedback will be disclosed. An example configuration that makes it possible to detect vital information with high accuracy and to perform proper heart rate variability biofeedback by setting a user face still and square with a camera will also be disclosed.

### (Configuration of Vital Information Display Apparatus)

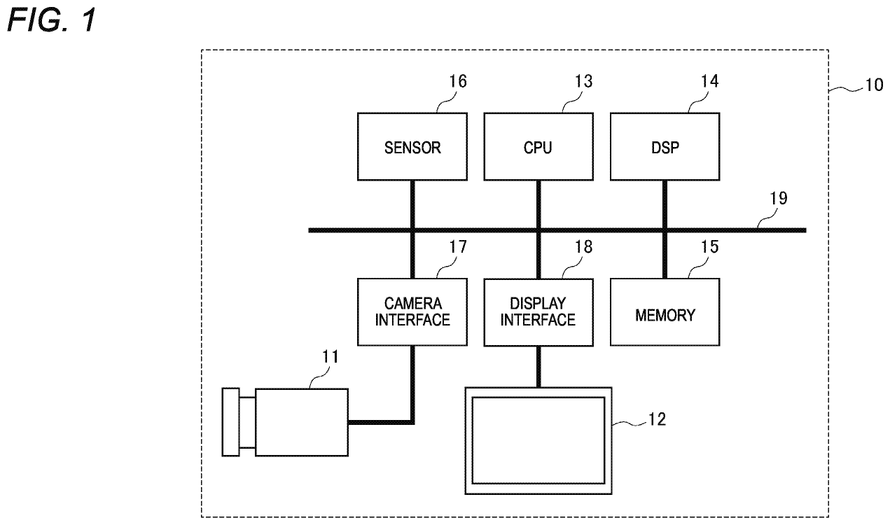

FIG. 1 is a block diagram showing an example configuration of a vital information display apparatus according to the embodiment. The vital information display apparatus 10 can be configured as an information processing apparatus such as a notebook PC (personal computer), a tablet terminal or a smartphone. The vital information display apparatus 10 includes an imaging unit, a processing unit, and a display unit. The imaging unit and the display unit may be either integrated with each other or provided as separate units that are combined with (attached to) each other.

The vital information display apparatus 10 is equipped with a camera 11 which is an example of the imaging unit, a monitor 12 which is an example of the display unit, a CPU (central processing unit) 13 and a DSP (digital signal processor) 14 which constitute the processing unit, and a memory 15. In the vital information display apparatus 10, the camera 11 and the monitor 12 are installed on the same surface in an apparatus body. The phrase “installed on the same surface” means that the camera 11 and the monitor 12 can be seen from a user. It suffices that the camera 11 and the monitor 12 be disposed on the same surface. They need not always be disposed in the same plane; for example, the camera 11 may be disposed at such a position as to project beyond the monitor 12. The vital information display apparatus 10 is also equipped with a sensor 16 for detecting the top-bottom direction of an image displayed on the monitor 12, a camera interface 17 for input/output of a signal taken by the camera 11 and a control signal, a display interface 18 for input/output of a video signal of the monitor 12 and a control signal, and a bus 19 that connects the individual units. In the vital information display apparatus 10, the CPU 13, the DSP 14, the memory 15, the sensor 16, the camera interface 17, and the display interface 18 are connected to each other by the bus 19, whereby various signals and data are exchanged during operation of the apparatus.

The camera 11 is equipped with an imaging lens and an imaging device such as a CCD (charge-coupled device) image sensor or CMOS (complementary metal-oxide-silicon) image sensor. The camera 11 images the face of an observation target person (i.e., user) and acquires and outputs video data of a prescribed detection area, including a skin portion, of the face. The camera 11 is connected to the CPU 13, the DSP 14, the memory 15, etc. via the camera interface 17 and outputs video data including the shot face portion of the user.

The monitor 12 is configured using a display device such as an LCD (liquid crystal display or an organic EL (electroluminescence) display. Connected to the CPU 13, the DSP 14, the memory 15, etc. via the display interface 18, the monitor 12 receives video data to be displayed on its screen and displays various kinds of information such as an information display picture (displayed during operation of the vital information display apparatus 10) and a setting picture. The monitor 12 displays, in the information display picture (displayed during operation), various kinds of information for heart rate variability biofeedback including a breathing pacer that gives an instruction relating to a breathing pace, an indicator indicating states of breathing and heart beats of the user (judgment results), vital information measurement results, etc. The camera 11 is installed in the vicinity of a side of the outer circumference of the information display screen of the monitor 12. The details of the information display picture will be described later.

The CPU 13, which is an example of a processor that performs various kinds of processing of individual units of the information processing apparatus, controls various operations for acquisition of vital information, display of vital information, monitor display for heart rate variability biofeedback, etc. The DSP 14, which is an example of a processor for performing data processing in the information processing apparatus, performs such processing as signal processing relating to vital information and image processing relating to monitor display. The memory 15, which is a storage device such as an SRAM (static random access memory) or a flash memory, stores various kinds of data such as operation programs of the apparatus, set information, information relating to components of the information display picture, and acquired vital information. The memory 15 also functions as a working memory for various kinds of processing performed during operation.

The sensor 16, which functions as an example detection unit is configured so as to have a 6-axis sensor, detects orientation and rotation of an apparatus body (terminal) including the camera 11 and the monitor 12 by detection of acceleration in 3-axis directions. The sensor 16 may be such as to detect a position of the camera 11. Configured in this manner, the sensor 16 can detect the top-bottom direction of the information display picture which is a display image of the monitor 12, a position of the camera 11 (i.e., a manner of disposition of the camera 11 with respect to the information display picture of the monitor 12), orientation of the monitor 12, a positional relationship between the camera 11 and the information display picture of the monitor 12, etc.

### (Detection of Vital Information)

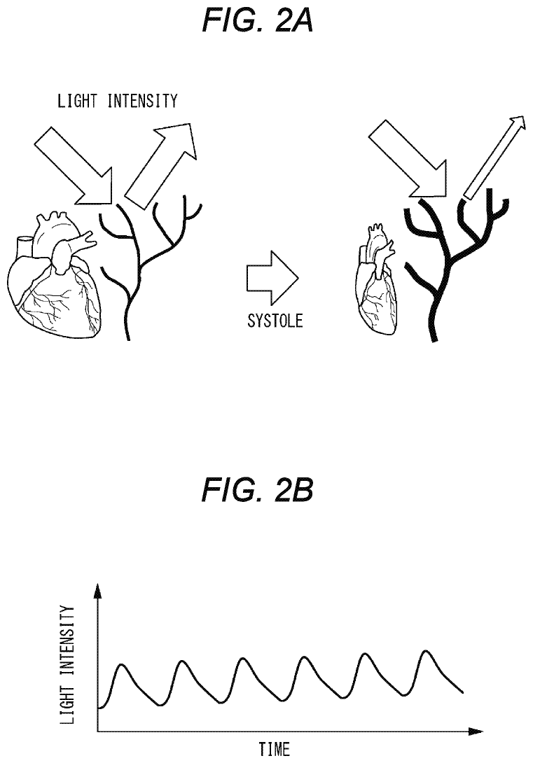

Now, the principle of detection of a pulse wave which is an example vital information in the vital information display apparatus according to the embodiment with reference to FIGS. 2A and 2B. FIG. 2A is a schematic diagram showing an example relationship between contraction of a human heart and the amount of absorption of light by blood vessels. FIG. 2B is a graph showing an example time-series variation of light intensity.

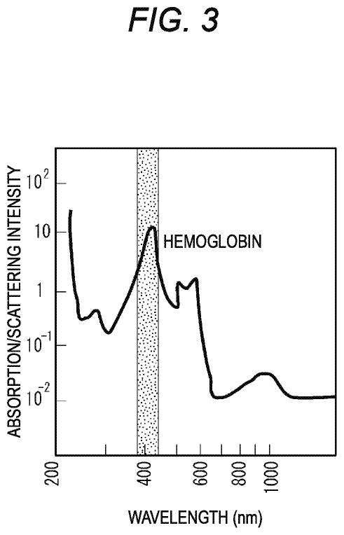

FIG. 2A shows how the capacity of blood vessels varies in synchronism of contraction of a human heart (systole). When the capacity of blood vessels increases as the heart contracts, the amount of absorption of light (e.g., light in a particular wavelength range as shown in FIG. 3) is increased and hence the light intensity is decreased (see FIG. 2B). A pulse wave indicates a movement of waves when pressure variations that occur in blood vessels due to pushing-out of blood into the main artery by systole propagate toward peripheries.

In FIG. 2B, the horizontal axis represents time and the vertical axis represents the intensity of a signal (photoelectric pulse wave) obtained by a variation of the light absorption amount. More specifically, referring to FIG. 2B, a peak appears in a state that the light absorption amount is small and the capacity of blood vessels is not increased and a minimum value appears in a state that the light absorption amount is large and the capacity of blood vessels is increased. The systole and the variation of the intensity of a photoelectric pulse wave are basically synchronized with each other though they are not completely synchronized with each other and a short delay exists because of distances between the heart and peripheral portions.

FIG. 3 is a graph showing an example of how the absorbance of hemoglobin varies depending on the wavelength of light. It is seen from FIG. 3 that hemoglobin (blood) tends to absorb light having a wavelength 400 nm (green light). Incidentally, although in the following embodiment will be described utilizing the fact that the absorbance of a green light component is high, a description may be made utilizing the fact that, for example, the reflectance of a red light component (e.g., longer than 1,000 nm in wavelength) is high.

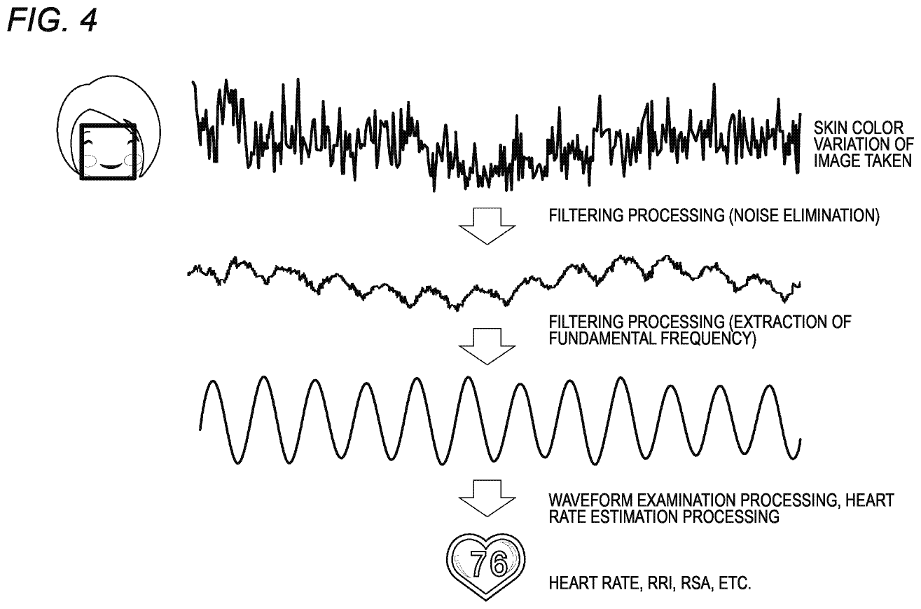

FIG. 4 is a diagram showing example of signals of vital information extracted from a video signal of an image taken. In the vital information display apparatus 10 according to the embodiment, a pulse wave is detected from a video of (moving image) of a skin portion of a detection area using video data of a user face acquired by the camera 11 (imaging unit) and vital information such as a heart rate is determined through calculation.

First, the DSP 14 of the processing unit receives video data of an image taken by the camera 11, extracts a face portion from the image taken by performing known face detection processing, and extracts a skin portion, having a skin color, of the face by recognizing the skin color in the face portion. The processing unit detects a pulse wave from the extracted skin portion.

In doing so, the DSP 14 of the processing unit constitutes a filter unit that has a bandpass filter having prescribed filter coefficients and performs filter processing on video data having a skin color variation. The filter unit eliminates a noise signal that was involved, for example, at the time of imaging by the camera 11 by averaging a signal (pixel values) in a prescribed range of the input video data. If a detection area of an image taken includes a human, this averaging can extract a human pulse wave. However, since it is highly probable that a human body movement component or a noise residual component still remains, the filter unit cuts out frequency components other than a fundamental frequency component of the pulse wave using set filter coefficients. The filter coefficients of the filter unit are set in advance so that signals of 30 to 120 bpm pass through the filter unit. Although in general the rest heart rate of adults is 60 to 80 bpm, here the pass range of the filter unit is set to 30 to 120 bpm taking into consideration influence of a case that an observation target person is tense.

The DSP 14 of the processing unit constitutes a waveform examination unit and a heart rate estimation unit and performs waveform examination processing and heart rate estimation processing on a fundamental frequency waveform of a pulse wave. To detect a noise signal interval that could not be cut out by the filter unit, the waveform examination unit judges whether there exists a signal interval that satisfies prescribed conditions of a noise signal using a signal of at least one cycle of an output signal of the filter unit. The waveform examination unit eliminates, as an invalid interval, an interval, judged to satisfy the prescribed conditions, of the output signal. For example, the waveform examination unit eliminates signals that are far larger or smaller than a prescribed value (e.g., 0). The processing performed by the waveform examination unit utilizes the knowledge that the amplitude of a human pulse wave varies gently within a prescribed range, and the probability that a signal that satisfies the prescribed conditions is disturbance noise is high.

The heart rate estimation unit calculates a human heart rate (pulse rate) on the basis of input intervals between frames of image data using a signal of at least one cycle as subjected to the filtering processing and the waveform examination processing. If the waveform examination unit has judged that there exists a signal interval that satisfies the prescribed conditions, the heart rate estimation unit calculates a heart rate using a signal of at least one cycle from which such a signal interval is eliminated. On the other hand, if the waveform examination unit has judged that there exists no signal interval that satisfies the prescribed conditions, the heart rate estimation unit calculates a heart rate using a signal itself of at least one cycle.

A signal as subjected to the filtering processing and the waveform examination processing has a cycle period that is equivalent to that of a signal component of a pulse wave. Thus, as shown in FIG. 4, a heart rate can be calculated and vital information such as a heartbeat interval (RRI: R-R interval) can be extracted by performing the filtering processing and the waveform examination processing on a signal representing a skin color variation of the image taken. Incidentally, a respiratory sinus arrhythmia (RSA) component, a Mayer wave-related sinus arrhythmia (MWSA) component, etc. can be calculated as vital information in addition to an RRI and heart rate variability biofeedback can be performed using such vital information.

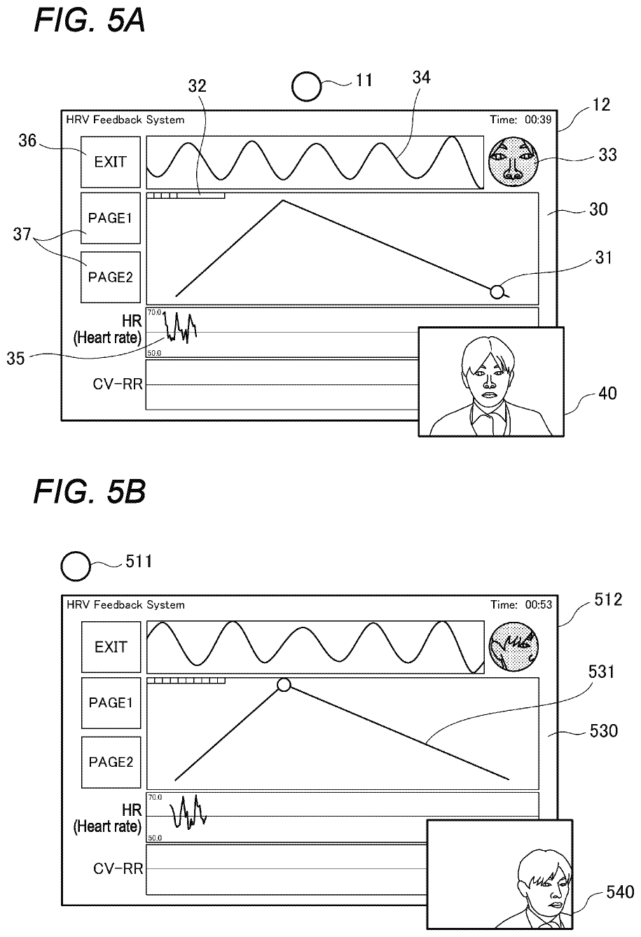

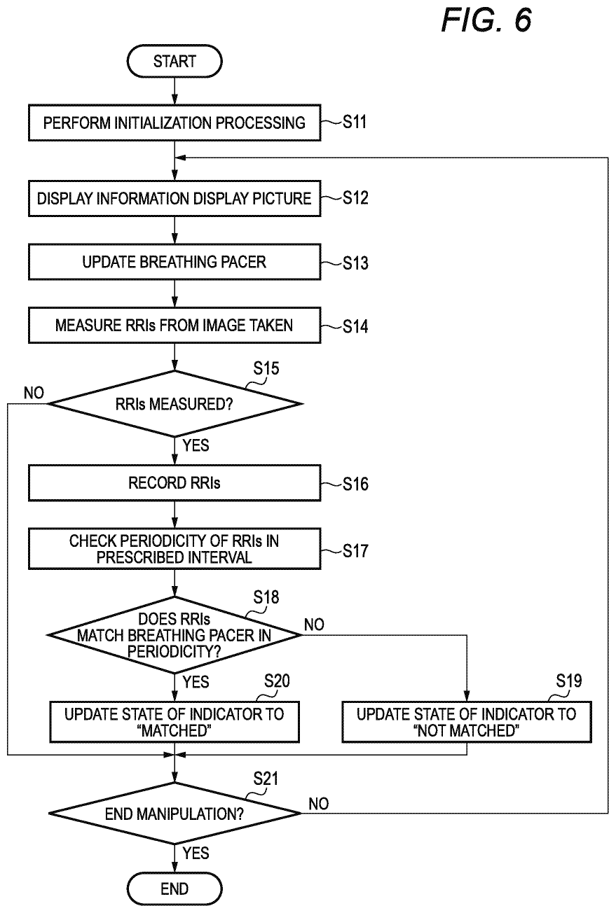

FIG. 5A is for description of an example (Display example-1) of the information display picture of the vital information display apparatus according to the embodiment. The example shown is of the vital information display apparatus which is a notebook PC.

In the vital information display apparatus 10, an information display picture 30 for heart rate variability biofeedback is displayed on the monitor 12. The camera 11 is disposed in the neighborhood of the monitor 12, that is, outside a central portion of a longer side of the outer circumference of the display screen of the monitor 12. The information display picture 30 has GUIs (graphical user interfaces) relating to heart rate variability biofeedback. The information display picture 30 includes, as part of the GUIs, a breathing assistant for assisting breathing. For example, the breathing assistant includes a breathing pacer 31 that gives an instruction relating to a breathing pace, an indicator 32 indicating states of breathing and heart beats of the user, etc. The breathing pacer 31 and the indicator 32 are provided in a region in the vicinity of the longer side that is closest to the camera 11, in the information display picture 30. The breathing assistant may be displayed in any of various display patterns such as only the breathing pacer, only the indicator, both of the breathing pacer and the indicator, and neither the breathing pacer nor the indicator. Even in the case where the display of the breathing assistant is turned off, the functions of the breathing pacer, the indicator, etc. continue to work. The information display picture 30 may include a face image display 33 of a face detection area of the user and a display 34 of variation components including a pulse wave extracted from an image taken. The information display picture 30 may also include detected various kinds of vital information 35, a stop button (EXIT) 36, and display switching buttons (PAGE1 and PAGE2) 37. The user can change the settings of the display form of the information display picture 30 in a desired manner. When the user has become accustomed to manipulation methods, it is possible to perform heart rate variability biofeedback even in a state one or both of the breathing pacer and the indicator are not displayed.

The breathing pacer 31 is, for example, a horizontally long GUI in the information display picture 30 and is a guide display that gives the user instructions as to a pace and timing of breathing-in and breathing-out by moving a circular mark (guide) along a triangular-mountain-shaped curve in its longitudinal direction. The indicator 32 is an index display indicating feedback information of a result of judgment as to whether heartbeats are occurring in proper rhythm in accordance with correlation between breathing and heartbeats of the user. For example, if it is judged that heartbeats are occurring in proper rhythm in accordance with correlation between breathing and heartbeats of the user, the display of the indicator 32 elongates gradually. The indicator 32 is an index display indicating feedback information of a result of judgment as to whether the user is breathing as instructed by the breathing pacer 31. For example, if the user is breathing as instructed by the breathing pacer 31, the display of the indicator 32 elongates gradually. The indicator 32 is, for example, a horizontally long GUI in the information display picture 30 and informs the user whether a heart rate variation is in a stable state in the form of a color, color percentages, or the like of a bar display. Where it is a bar display, the indicator 32 may be a display using a state variation such as a color variation of the bar, a display in which the length of a (colored) bar is changed in the longitudinal direction, or the like. The display colors of the indicator 32 are selected so as to allow the user to recognize a heart rate variation state intuitively at a glance, for example, by displaying an unstable heart rate variation state by red, displaying a state that the heart rate variation has changed from an unstable state to a little stable state by yellow, displaying a state that the heart rate variation has changed to a further stable state by blue, and displaying a state that the heart rate variation is stable by green. The length of the bar is changed in such a manner that, for example, the bar is short in a state that the heart rate variation is unstable, is elongated as the heart rate variation becomes more stable, and is kept long in a state that the heart rate variation is stable.

As the user receives heart rate variability biofeedback according to instructions of the breathing pacer 31, the display color of the indicator 32 is changed so as to indicate more stable state increasingly, for example, in order of red→yellow→blue→green. The heart rate variation becomes large when psychological stress is low and the activity of the parasympathetic nerves of the autonomic nervous system becomes dominant. In this state, RRI fluctuations become large and, in general, RRI values become large.

The face image display 33 is a display of a detection area of a face in an image taken and, for example, a grid-like portion in the figure represents a skin portion of the face. Vital information such as an RRI can be detected more accurately when the area of the skin portion is larger in the face image display 33. For example, an RRI can be calculated from an interval between mountain peaks of the waveform of the variation component display 34 shown in the figure.

In the embodiment, the camera 11 is located adjacent to a central portion of a longer side of the information display picture 30 of the monitor 12. And, in the information display picture 30, the breathing pacer 31 and the indicator 32 are located in a region that is in the vicinity of the longer side that is closest to the camera 11. With these measures, during imaging by the camera 11, the angle of view becomes suitable for acquisition of vital information by imaging of the user face. Thus, when the user directs his or her face to the breathing pacer 31 to see it to receive heart rate variability biofeedback, the user face is shot naturally (automatically) so as to be located at and with such a imaging position and an angle that are suitable for measurement of a heart rate using an image taken by the camera 11. Furthermore, the size of the user face portion in an image taken can be set at a proper size by changing the positional relationship and the distance between the camera 11 and the breathing pacer 31 etc. in the information display picture 30. Still further, in the information display picture 30, the breathing pacer 31 and the indicator 32 are disposed in such ranges as to allow the user to see them merely by moving his or her line of sight or moving his or her line of sight as little as possible.

For example, the locations of the breathing pacer 31 and the indicator 32 in the information display picture 30 are set according to a position of the camera 11, orientation of the monitor 12 and the information display picture 30, a relative positional relationship between the camera 11 and the information display picture 30, and other things as detected by the sensor 16. A position of the camera 11 and a positional relationship between the camera 11 and the information display picture 30 of the monitor 12 can be detected on the basis of any of information relating to initial settings made by the user, information acquired from an OS (operating system) that enables operation of the apparatus, information as default settings of the apparatus, information acquired from terminal information of the apparatus, and other kinds of information.

Thus, like an image 40 taken that is shown at a bottom-right position in FIG. 5A, when the user is looking at the breathing pacer 31 or the indicator 32, a face image taken almost squarely by the camera 11 can always be obtained. This facilitates detection of a user face and makes it possible to acquire a large skin portion, having a skin color, of the face stably. Furthermore, a movement of the user face can be suppressed and, even if the face is moved, the angular variation with respect to the camera 11 is so small that the influence of a face movement can be made small. As a result, heartbeat disorder at the time of acquisition of a heart rate from an image taken can be suppressed, whereby vital information relating to a heart rate variation can be acquired stably and the accuracy of detection of vital information can be increased. Furthermore, the apparatus is made easier to use because it performs heart rate variability biofeedback by detecting vital information without making contact using an image taken

It is preferable that the camera 11 so as to be disposed in the vicinity of the outer circumference of the monitor 12 so as to be integrated with it in the apparatus body as in a notebook PC, a tablet terminal, a smartphone, or the like. In this case, the camera 11 is disposed over or under a central portion of the information display picture 30. From the viewpoints of the appropriateness of the angle of view at the time of taking of a face image by the user and the stability of detection of a skin portion, it is even preferable that the camera 11 be disposed over a central portion of the information display picture 30. In the information display picture 30, main GUIs such as the breathing pacer 31 and the indicator 32 are disposed in a region that is closest to the camera 11, that is, in a central region in the left-right direction of the screen and in a top region in the top-bottom direction. Whether the information display picture 30 should be long in the horizontal direction or the vertical direction is determined according to the position of the camera 11 and the top-bottom direction of the information display picture 30. With these measures, when heart rate variability biofeedback is performed, the user can see the breathing pacer 31 and the indicator 32 merely by moving his or her line of sight as little as possible.

The camera 11 may be provided separately from the monitor 12 and attached to it. In this case, the camera 11 is attached to the monitor 12 using an attachment member such as a bracket so as to be located in the vicinity of the monitor 12. The positional relationship between the camera 11 and the information display picture 30 of the monitor 12 is the same as in the case where they are disposed so as to be integrated with each other; the main GUIs such as breathing pacer 31 and the indicator 32 are disposed in regions close to the camera 11.

Where the camera 11 is not located adjacent to a central portion of the information display picture 30, that is, it is deviated leftward or rightward, the main GUIs such as breathing pacer 31 and the indicator 32 in the information display picture 30 are deviated so as to be located in regions close to the camera 11. A further alternative configuration is possible in which the camera 11 is deviated sideways (leftward or rightward) from a central portion of the information display picture 30 of the monitor 12 and the imaging optical system of the camera 11 is given a lens shift function so that an image as obtained when the user face is shot squarely can be taken by imaging from an oblique direction.

Location information of the camera 11 may be acquired by any of various methods such as that its position is set as an initial setting by the user, that position information corresponding to a type of an apparatus terminal is acquired, that position information is acquired from setting information of the OS, and that position information a preset, default setting is used. Where camera position information corresponding to a type of an apparatus terminal is acquired, it is possible to, for example, acquire camera position information of the type concerned by referring to terminal information (type information) of the apparatus and a database of camera positions of respective terminal types.

The manner of expressing the breathing pacer 31 and the indicator 32 is not limited to the case that they are expressed as separate GUI displays and they may be expressed as an integrated GUI display. For example, it is possible to express the breathing pacer in the form of a GUI movement or a size variation and to express the indicator as a GUI color variation. The breathing pacer may be configured in such a manner that the vital information display apparatus is provided with a sound generation unit such as a speaker and the user is given an instruction relating to breathing timing using a sound generated from the sound generation unit in addition to a display in the information display picture 30. The indicator may be configured in such a manner that only the breathing pacer is displayed in the information display picture and the user is informed of a breathing variation state using a sound generated from the sound generation unit.

FIG. 5B is for description of an example of the information display picture of Comparative Example. In contrast to the display example employed in the embodiment shown in FIG. 5A, in Comparative Example shown in FIG. 5B, a camera 511 is disposed outside a left end portion of a longer side of an information display picture 530 of the monitor 512. In this case, a breathing pacer 531 is distant from the camera 511. As a result, the user face is shot obliquely like an image 540 taken that is shown at a bottom-right position in the figure and hence it is difficult to perform face detection and a skin portion, having a skin color, of the face in the image taken becomes small. Furthermore, while the user is seeing the breathing pacer or the indicator, a large face movement is caused by a movement of the line of sight in the left-right direction or the top-bottom direction. Still further, a large angular variation occurs with respect to the camera 511 when the face is moved, making the influence of the face movement large. Thus, in this Comparative Example, there may occur an event that vital information relating to a heart rate variation cannot be acquired stably due to disorder in the vital information. This means a problem that heart rate variability biofeedback cannot be performed in a desired manner depending on the method of non-contact detection.

FIG. 6 is a flowchart showing a processing procedure to be followed in a heart rate variability biofeedback operation performed in the embodiment. Heart rate variability biofeedback is performed as a prescribed program is run by the CPU 13 of the vital information display apparatus 10 and various steps are thereby executed being mainly controlled by the CPU 13.

Upon a start of a heart rate variability biofeedback operation, the vital information display apparatus 10 performs various kinds of initialization processing relating to this operation (S11). In the initialization processing, the vital information display apparatus 10 initializes parameters of a breathing pacer 31 and an indicator 32 to be displayed in an information display picture 30. In doing so, the vital information display apparatus 10 sets the parameters of the indicator to a state “non-coincidence” meaning that the heart rate is not stable, sets the parameters of the breathing pacer to initial states (start position and start state), and clears RRI data that have been stored so far in the memory 15.

After the initialization processing, the vital information display apparatus 10 displays an information display picture 30 including the breathing pacer 31 and the indicator 32 on the monitor 12 as GUIs relating to heart rate variability biofeedback (S12). The vital information display apparatus 10 starts imaging the face of a user who is facing the information display picture 30 by the camera 11 which is located in the vicinity of the monitor 12 and acquires, continuously, a moving image taken that includes a skin portion of the face.

Then the vital information display apparatus 10 gives the user instructions relating to breathing timing (breathing-in and breathing-out timing) by updating the breathing pacer 31 in the information display picture 30 when necessary and thereby moving the breathing pacer 31 with prescribed cycle and timing (S13). The user breathes following the instructions of the breathing pacer 31 while locking at the breathing pacer 31 displayed in the information display picture 30.

The vital information display apparatus 10 calculates and acquires vital information from video data of a moving image taken through signal processing that is performed by the DSP 14. At this step, the vital information display apparatus 10 measures a heart rate by measuring RRIs as vital information through calculation (S14). Then the vital information display apparatus 10 judges whether RRIs have been measured normally (S15). If RRIs have not been measured normally or updated, the vital information display apparatus 10 moves to end judgment processing (S21). If RRIs have been measured normally, the vital information display apparatus 10 records RRI data acquired sequentially in the memory 16 (S16).

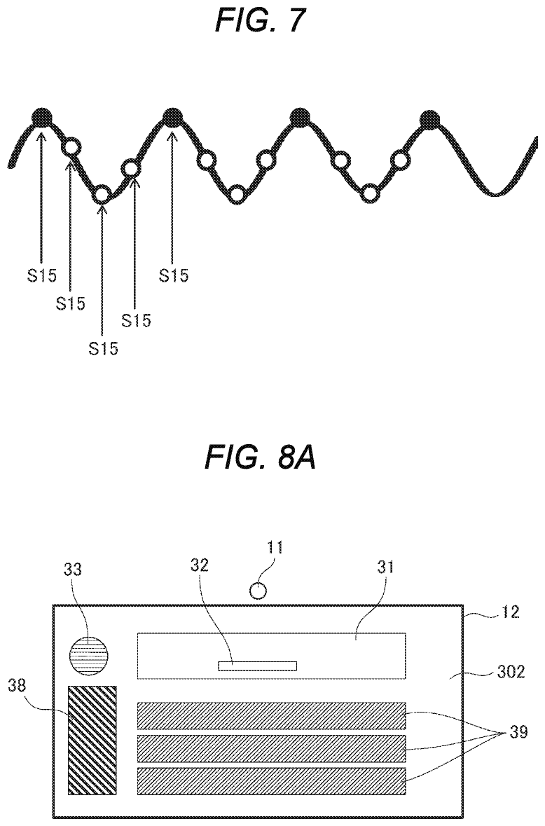

FIG. 7 is a diagram for description of RRI output timing. Assume that filtering processing and waveform examination processing have been performed on a skin color variation signal of an image taken and a signal waveform shown in FIG. 7 that is equivalent in cycle period as a pulse wave of an observation target person has been obtained. The DSP 14 is configured so as to output an RRI when the signal waveform has a maximum value and each of points indicated by white circles and black circles indicates timing of making an RRI measurement judgment of S15 (see FIG. 6). The judgment result at step S15 is “yes” at each peak point (maximum point) indicated by a black circle of the signal waveform, and is “no” at each point indicated by a white circle because no RRI can be measured. In this case, the DSP 14 updates the RRI sequentially at the timing of the maximum points (black circles).

Subsequently, the vital information display apparatus 10 calculates a fluctuation of the RRI data recorded in the memory 15 and checks regularity (periodicity) of RRIs in a prescribed interval (S17). In doing so, to produce a vital information judgment result, the vital information display apparatus 10 judges whether the RRIs match the breathing pacer in periodicity (S18). Whether the RRIs match the breathing pacer in periodicity is judged by, for example, judging whether the difference in, for example, cycle period or fluctuation, between the RRIs and the breathing pacer is smaller than or equal to a prescribed threshold value. Such periodicity matching judgment makes it possible to judge whether heartbeats and breathing of the user are in harmony, that is, whether psychological stress is low and the heart rate is stable.

If judging that the RRIs do not match the breathing pacer in periodicity, the vital information display apparatus 10 updates the state of the indicator 32 in the information display picture 30 to “not matched” (S19). Since the state of the indicator 32 is “not matched” in the initial state, “not matched” is maintained for a while after the start of the operation. If judging that the RRIs match the breathing pacer in periodicity, the vital information display apparatus 10 updates the state of the indicator 32 in the information display picture 30 to “matched” (S20). That is, the state of the indicator 32 is changed to “matched” when the measured RRIs of the user match the breathing pacer displayed on the screen in periodicity.

Then the vital information display apparatus 10 judges whether an end manipulation has been made by, for example, pushing the stop button 36 (S21) and finishes the operation if an end manipulation has been made. If no end manipulation has been made yet, the vital information display apparatus 10 returns to step S12 (the step of displaying the information display picture) and repeats the above-described heart rate variability biofeedback processing. The vital information display apparatus 10 repeats these steps until an end manipulation is made.

### (Other Display Examples of Information Display Picture)

Several other display examples of the information display picture will be described below. The proper arrangement of the GUIs displayed in the information display picture 30 such as the breathing pacer 31 and the indicator 32 depend on the positional relationship between the monitor 12 and the camera 11 and the orientation of the information display picture 30. In view of this, a description will be made of how the GUIs employed in the embodiment are arranged according to various sets of conditions.

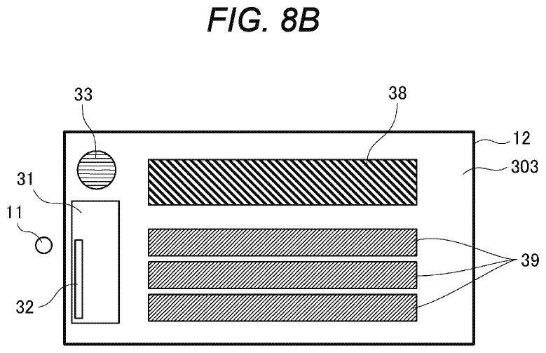

FIG. 8A is a diagram showing Display example-2 of the information display picture employed in the embodiment. In Display example-2, the camera 11 is disposed outside a central portion of the top longer side of the outer circumference of the display screen of the monitor 12. An information display picture 302 displayed on the monitor 12 is oblong (the longer sides are in the horizontal direction). In the information display picture 302, an oblong breathing pacer 31 and indicator 32 are disposed in a region in the vicinity of the longer side that is closest to the camera 11, that is, in a top-center region of the information display picture 302. A face image display 33 is disposed on the left of the breathing pacer 31 and a menu display 38 such as a stop button and display switching buttons is disposed in a vertically long region that is located under the face image display 33, that is, in a left end region of the screen. Acquired data displays 39 such as a variation components display including a pulse wave and various vital information displays are disposed in regions located under the breathing pacer 31 and the indicator 32. For example, various kinds of image data such as a skin color average value acquired from an image taken of a user face portion, a heart rate (BR), history information of a heart rate variation (RRIs), various vital indices (CV-RR, SDNN, rMSSD, LF/HF, tone, entropy, etc.), and a Lorentz-plot curve can be displayed as the acquired data displays 39.

FIG. 8B is a diagram showing Display example-3 of the information display picture employed in the embodiment. In Display example-3, the camera 11 is disposed outside a central portion of the left shorter side of the outer circumference of the display screen of the monitor 12. An information display picture 303 displayed on the monitor 12 is oblong (the longer sides are in the horizontal direction). In the information display picture 303, an vertically long breathing pacer 31 and indicator 32 are disposed in a region located in the vicinity of the shorter side that is closest to the camera 11, that is, in a center-left region of the information display picture 303. A face image display 33 is disposed over the breathing pacer 31 and a menu display 38 is disposed in a horizontally long region that is located on the right of the face image display 33, that is, in a top region of the screen. Acquired data displays 39 are disposed in regions located on the right of the breathing pacer 31 and the indicator 32.

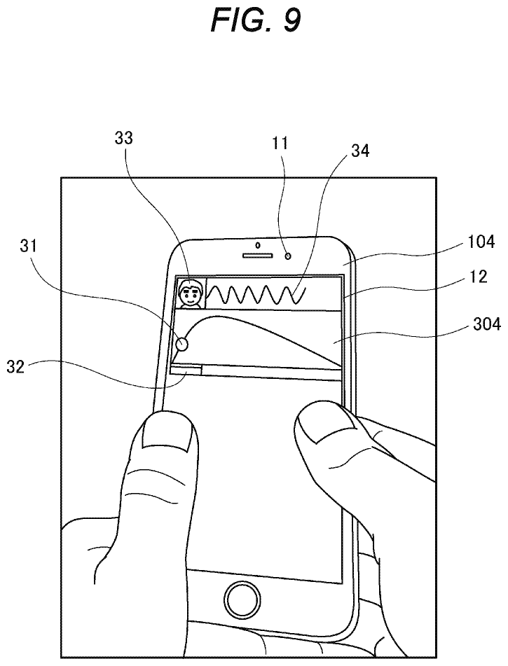

FIG. 9 is a view showing Display example-4 of the information display picture employed in the embodiment. Display example-4 is a display example of a case that a vital information display apparatus is configured in the form of a smartphone which is a communication terminal. In the vital information display apparatus 104, a monitor 12 which is shaped like a vertically long rectangle is provided in a vertically long body having rounded corners and a camera 11 for imaging the face of the user himself or herself is disposed outside (i.e., over) a shorter side of the outer circumference of the monitor 12 at a position that is deviated a little rightward from the center in the horizontal direction. An information display picture 304 displayed on the monitor 12 is vertically long, that is, its longer sides are vertical. A horizontally long breathing pacer 31 and indicator 32 are disposed in a region located in the vicinity of the shorter side that is closest to the camera 11 in the information display picture 304, that is, in a top region in the information display picture 304. A face image display 33 and a variation component display 34 are disposed over the breathing pacer 31. In this case, even if the user holds the apparatus body firmly with, for example, both hands to keep the apparatus body as still as possible to measure a heart rate stably, the breathing pacer 31 and the indicator 32 are displayed without being hidden because they are located in a top portion of the monitor 12.

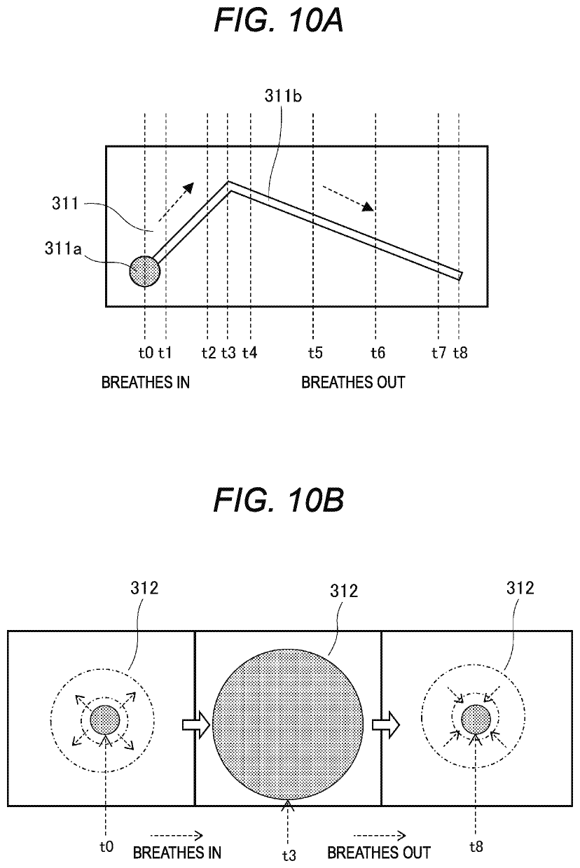

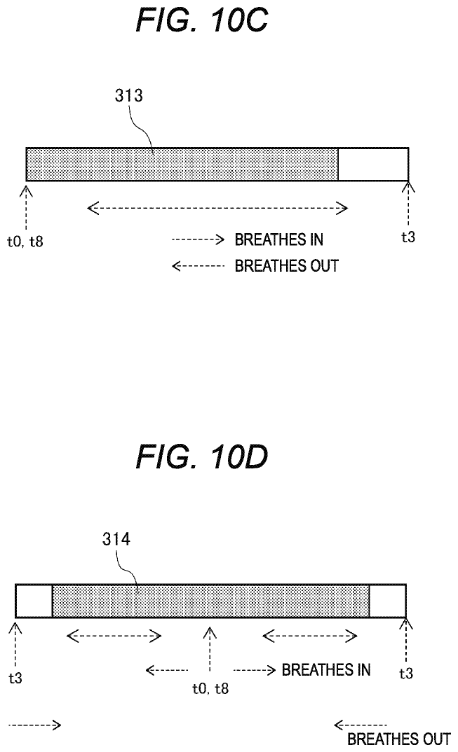

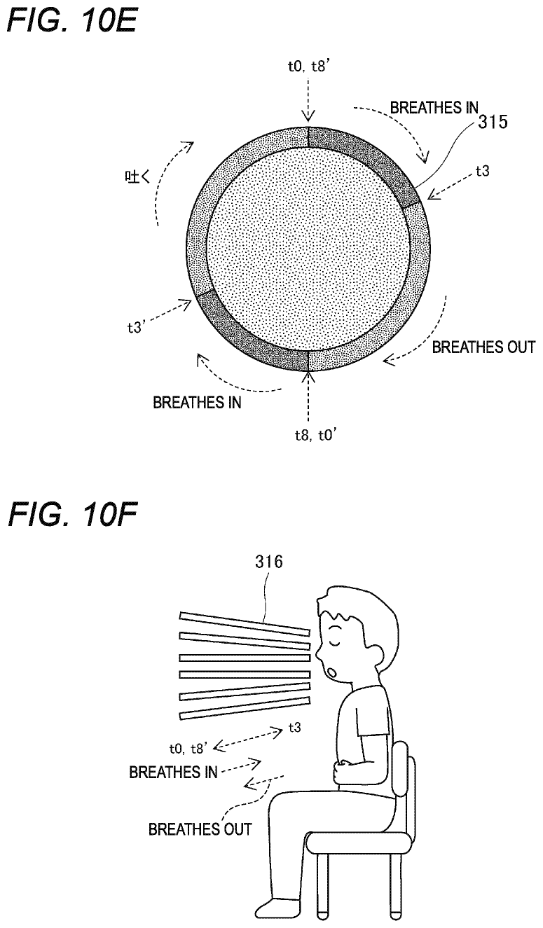

FIG. 10A is a diagram showing Display example-1 of the breathing pacer displayed in the information display picture employed in the embodiment. Several specific examples of the breathing pacer displayed in the information display picture will be described below. A breathing pacer 311 of Display example-1 is to instruct the user on a breathing pace and timing in such a manner that a circular mark 311a as a guide moves along a triangular-mountain-shaped curve 311b. In the illustrated example, for convenience of description, individual times t1-t8 are shown in the breathing pacer 311 as indices in one cycle of breathing. The times t1-t8 are not shown when the breathing pacer 311 is displayed actually. The time intervals between times t0 and t1, t1 and t2, . . . , t7 and t8 are the same in length.

The interval t0 to t3 which is an uphill of the curve 311b of the breathing pacer 311 is a breathing-in interval. The mark 311a of the breathing pacer 311 stops at time t0, accelerates from time t0 to t1, moves at a constant speed from time t1 to t2 (i.e., after the acceleration), and decelerates from time t2 to t3; in this manner, the mark 311a starts from the stop position, accelerates gradually, moves at a constant speed, and then decelerates, thus finishing the breathing-in period (interval). For example, the mark 311a accelerates from time t0 to t1 so as to reach a speed at which to move from time t1 to t2 and decelerates from time t2 to t3 from the speed at which it moved from time t1 to t2. The mark 311a may stop at time t3 which is a breathing-in/breathing-out switching time point. On the other hand, the interval t3 to t8 which is a downhill of the curve 311b of the breathing pacer 311 is a breathing-out interval. The mark 311a of the breathing pacer 311 accelerates from time t3 to t4, moves at a constant speed from time t4 to t4, from t5 to t6, and from t6 to t7, decelerates from time t7 to t8, and stops at time t8; in this manner, the mark 311a accelerates gradually from a low-speed or stopped state, moves at a constant speed, then decelerates and stops, thus finishing the breathing-out period (interval). For example, the mark 311a accelerates from time t3 to t4 so as to reach a speed at which to move from time t4 to t5 and decelerates from time t7 to t8 from the speed at which it moved from time t6 to t7. As described above, in the breathing pacer 311, the movement speed of the mark 311a which is to serve as a guide varies as it moves in the individual intervals according to breathing timing.

Displaying the breathing pacer in which as described above the mark moves at prescribed speeds that depend on the respective intervals allows the user to follow the breathing pacer with his or her eyes easily and to adjust his or her breathing pace and timing so that they match the movement of the breathing pacer. Thus, the heart rate variability biofeedback can be performed properly.

FIG. 10B is a diagram showing Display example-2 of the breathing pacer displayed in the information display picture employed in the embodiment. A breathing pacer 312 of Display example-2 is to instruct the user on a breathing pace and timing by changing the size of a circular mark. In the illustrate example, marks corresponding to respective times t0 (start of breathing-in), t3 (switching from breathing-in to breathing-out), and t8 (end of breathing-out) are arranged in the horizontal direction. In an actual manner of display of the breathing pacer 312, the size of one circular mark varies in order of small→large→small, that is, the size of the circular mark increases in a breathing-in interval and decreases in a breathing-out interval. Alternatively, the size of the circular mark may vary in order of large→small→large. As a further alternative, the breathing pacer may be given an indicator function by, for example, changing the color of its mark so as to be able to also serve as an indicator. Since as described above the breathing pacer is displayed the size of whose mark changes at a prescribed rate, the movement of the breathing pacer allows the user to adjust his or her breathing pace and timing properly.

FIG. 10C is a diagram showing Display example-3 of the breathing pacer displayed in the information display picture employed in the embodiment. A breathing pacer 313 of Display example-3 is to instruct the user on a breathing pace and timing by changing the length of a bar. In the illustrated example, the breathing pacer 313 is a bar display that is long in the horizontal direction. In a breathing-in interval from time t0 to t3, the bar elongates gradually toward the right end gradually (left end is fixed) from a zero or short-length state. In a breathing-out interval from time t3 to t8, the bar shortens gradually from the right end (long state) toward the left end. Alternatively, the breathing pacer 313 may be a bar display that is long in the vertical direction (i.e., rotated). As a further alternative, the breathing pacer may be given an indicator function by, for example, changing the color of its mark so as to be able to also serve as an indicator. Since as described above the breathing pacer is displayed the length of whose bar changes at a prescribed rate, the movement of the breathing pacer allows the user to adjust his or her breathing pace and timing properly.

FIG. 10D is a diagram showing Display example-4 of the breathing pacer displayed in the information display picture employed in the embodiment. A breathing pacer 314 of Display example-4, which is a modified version of Display example-3, is to instruct the user on a breathing pace and timing in the form of a horizontal or vertical bar display by changing the length of a bar in such a manner that the bar contracts or expands toward the center or both ends. In the illustrated example, the breathing pacer 314 is a bar display that is long in the horizontal direction. In a breathing-in interval from time t0 to t3, the bar elongates gradually toward the left and right ends from the center from a zero or short-length state. In a breathing-out interval from time t3 to t8, the bar shortens gradually from both ends (long state) toward the center. Alternatively, the breathing pacer 314 may be a bar display that is long in the vertical direction (i.e., rotated). As a further alternative, the breathing pacer may be given an indicator function by, for example, changing the color of its mark so as to be able to also serve as an indicator. Since as described above the breathing pacer is displayed the length of whose bar changes at a prescribed rate, the movement of the breathing pacer allows the user to adjust his or her breathing pace and timing properly.

FIG. 10E is a diagram showing Display example-5 of the breathing pacer displayed in the information display picture employed in the embodiment. A breathing pacer 315 of Display example-5 is to instruct the user on a breathing pace and timing by moving an index such as a color band or mark along the circle in a ring-shaped display region as in a clock. In the illustrated example, the breathing pacer 315 makes a breathing instruction of one cycle per half circle, that is, has two sets of a breathing-in interval and a breathing-out interval per circle. Alternatively, a pace display for one set of breathing-in and breathing-out may be made per one time of circling. The breathing pacer 315 shows breathing-in intervals by causing the index of the ring to move clockwise along the circle from time t0 to t3 and from time t0′ to t3′, and shows breathing-in intervals by causing the index of the ring to move clockwise along the circle from time t3 to t8 and from time t3′ to t8′. Since as described above the breathing pacer is displayed in such a manner that the index moves along the circle at a prescribed speed, the movement of the breathing pacer allows the user to adjust his or her breathing pace and timing properly.

FIG. 10F is a diagram showing Display example-6 of the breathing pacer displayed in the information display picture employed in the embodiment. A breathing pacer 316 of Display example-6 is to instruct the user on a breathing pace and timing by an animation display that schematically shows how a person breathes. In the illustrated example, the breathing pacer 316 shows breathing-in intervals and breathing-out intervals by changing a human picture and plural radial lines. For example, in a breathing-in interval from time t0 to t3, the color of the lines changes or the lines shorten from the outside toward the person. In a breathing-out interval from time t3 to t8, the color of the lines changes or the lines elongate outward from the person. The chest and the belly of the person may be moved according to breathing-in intervals and breathing-out intervals. Since as described above the breathing pacer is displayed in the form of an animation in such a manner that its state is changed, the movement of the breathing pacer allows the user to adjust his or her breathing pace and timing properly.

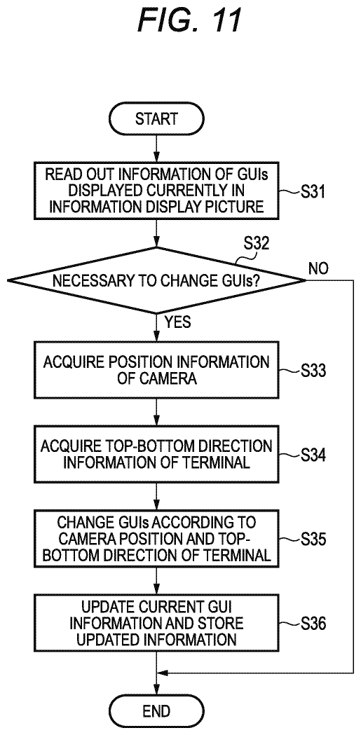

FIG. 11 is a flowchart showing a processing procedure to be followed when the orientation of the information display picture for heart rate variability biofeedback in the embodiment has been changed.

A description will be made of an example of process that is performed in the embodiment when the camera 11 and the monitor 12 of the vital information display apparatus 10 have been rotated and the orientation of the information display picture 30 has been changed because, for example, the user has moved the apparatus body. Individual steps of a display control to be performed in association with a change of the orientation of the information display picture 30 are executed mainly by controls performed by the CPU 13 of the vital information display apparatus 10 as the CPU 13 runs a prescribed program. The display control process shown in FIG. 11 is activated and started in response to detection of a change of the orientation of the information display picture 30 in such a manner that, for example, the process is called as an interrupt process by the CPU 13 when rotation of the monitor 12 is detected by the sensor 16.

The vital information display apparatus 10 reads out, from the memory 15, information of individual GUIs displayed currently in the information display picture 30 (S31). The vital information display apparatus 10 acquires, as GUI information, parameters etc. indicating a type, location, direction, size, and state of each GUI.

Then the vital information display apparatus 10 judges whether it is necessary to change the GUIs displayed currently in the information display picture 30 (S32). The vital information display apparatus 10 detects the top-bottom direction of the monitor 12 using the sensor 16 and judges whether to change the GUIs on the basis of the orientation, with respect to the user, of the information display picture 30 displayed on the monitor 12 and the positional relationship between the camera 11 and the information display picture 30 displayed on the monitor 12. The orientation of the information display picture 30 can be detected by another method such as detecting the direction of the user face from an image taken by the camera 11. The positional relationship between the camera 11 and the information display picture 30 displayed on the monitor 12 can be detected on the basis of any of information of an initial setting made by the user, information acquired from the OS that enables operation of the apparats, information that is set as default information of the apparatus, information acquired from terminal information of the apparatus, etc.

If it is necessary to change the GUIs displayed currently in the information display picture 30, first, the vital information display apparatus 10 acquires information indicating a position of the camera 11 with respect to the monitor 12 (S33). As for the position information of the camera 11, a physical position such as “above,” “below,” “right,” or “left” with respect to the monitor 12 provided in the apparatus body (terminal) is acquired. Furthermore, the vital information display apparatus 10 acquires top-bottom direction information of the apparatus body (terminal) (S34). Then the vital information display apparatus 10 changes the GUIs so that they become suitable for the camera position and the top-bottom direction of the terminal (i.e., the orientation of the information display picture 30) on the basis of the position information of the camera 11 and the top-bottom direction information of the terminal (S35).

After changing the GUIs displayed in the information display picture 30, the vital information display apparatus 10 updates the information of each GUI currently displayed in the information display picture 30 and stores the updated information of each GUI such as the orientation of the information display picture 30 and the direction of each GUI (S36), in the memory 15. Then the vital information display apparatus 10 finishes this process. If it is not necessary to change the GUIs displayed in the current information display picture 30, the vital information display apparatus 10 finishes this process leaving the GUIs as they are.

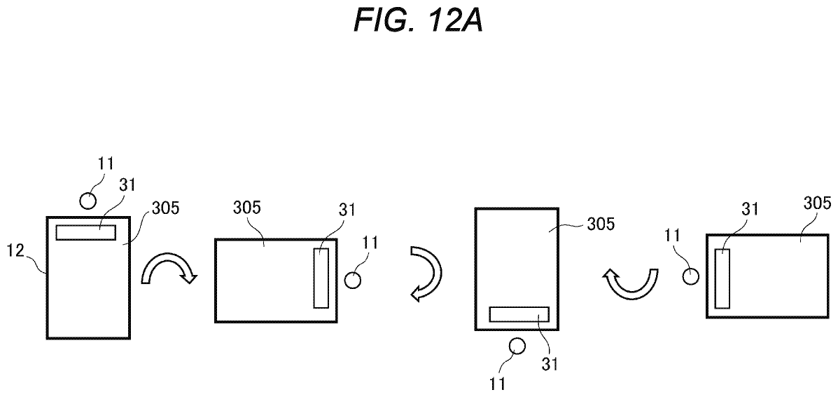

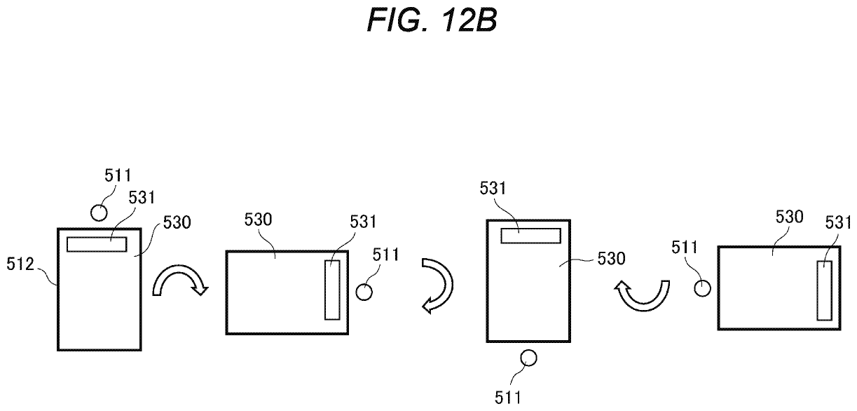

FIG. 12A is a diagram for description of an example (Display example-5) of the information display picture of the vital information display apparatus according to the embodiment. In the illustrated example, the camera 11 and the monitor 12 are rotated clockwise by 90° each time to assume a first state, a second state, a third state, and a fourth state (shown rightward in the figure) in this order.

In Display example-5, in the first state shown at the leftmost position in the figure, the camera 11 is disposed outside a central portion of the top shorter side of the outer circumference of the display screen of the monitor 12. An information display picture 305 displayed on the monitor 12 is vertically long and the loner sides are in the vertical direction. In this case, a horizontally long breathing pacer 31 is disposed in a region that is in the vicinity of the longer side that is closest to the camera 11, that is, in a top-center region of the information display picture 305. In the second state obtained by rotating the apparatus body by 90° clockwise, as shown at the second left position in the figure, the camera 11 is disposed adjacent to a center-right portion of the information display picture 305. In this case, because of the rotation of the camera 11 and the monitor 12, a vertically long breathing pacer 31 is disposed in a center-right region of the information display picture 305 so as to be suitable for the position of the camera 11.

In the third state obtained by rotating the apparatus body by 90° clockwise from the second state, as shown at the third left position in the figure, the camera 11 is disposed adjacent to a bottom-center portion of the information display picture 305. In this case, because of the rotation of the camera 11 and the monitor 12, a horizontally long breathing pacer 31 is disposed in a bottom-center region of the information display picture 305 so as to be suitable for the position of the camera 11. Furthermore, in the fourth state obtained by rotating the apparatus body by 90° clockwise from the third state, as shown at the fourth left position (rightmost position) in the figure, the camera 11 is disposed adjacent to a center-right portion of the information display picture 305. In this case, because of the rotation of the camera 11 and the monitor 12, a vertically long breathing pacer 31 is disposed in a center-left region of the information display picture 305 so as to be suitable for the position of the camera 11.

FIG. 12B is a diagram for description of an example of the information display picture of Comparative Example. In contrast to the display example according to the embodiment shown in FIG. 12A, in Comparative Example shown in FIG. 12B, the position of a breathing pacer 531 in an information display picture 530 is not changed even if a camera 511 and a monitor 512 are rotated. In this case, even if the camera 511 and the monitor 512 are rotated so as to assume a first state, a second state, a third state, and a fourth state in this order, the position of the breathing pacer 531 in the information display picture 530 is fixed. Thus, as in the third state that is shown at the third left position in the figure and the fourth state that is shown on the rightmost position in the figure, the breathing pacer 531 is located at a position that is distant from the camera 511. As a result, the user face that is directed obliquely may appear in an image taken or the user face may move when heart rate variability biofeedback is performed, possibly resulting in trouble that vital information cannot be obtained stably.

### (Examples of Arrangement GUIs in Information Display Picture)

Several examples of the arrangement of GUIs in which a change in the orientation of the information display picture is taken into consideration will be described. In these examples, it is assumed that GUIs having various shapes such as the breathing pacer 31 and the indicator 32 are arranged and how the GUIs are arranged according to various sets of conditions will be described.

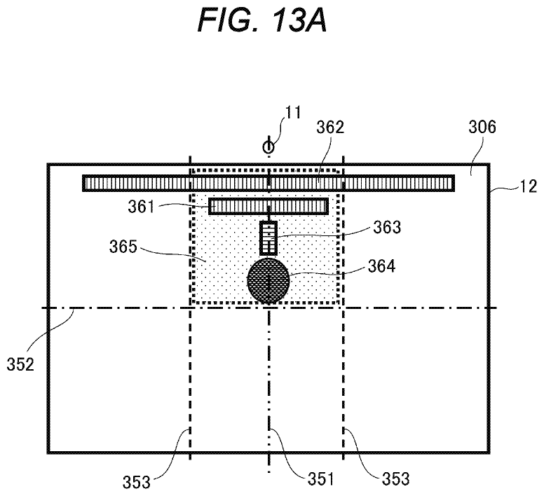

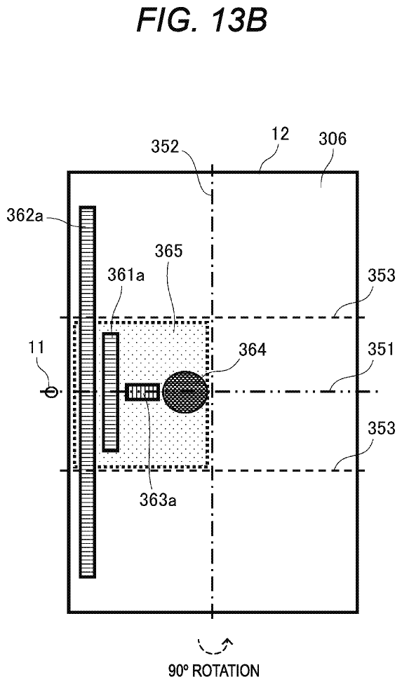

FIGS. 13A and 13B are diagrams showing Arrangement example-1 of GUIs in the information display picture employed in the embodiment. In Arrangement example-1 shown in FIG. 13A, the camera 11 is disposed outside (over) a central portion of the top longer side of the outer circumference of the display screen of the monitor 12. An information display picture 306 displayed on the monitor 12 is long in the horizontal direction (i.e., its longer sides are horizontal). In the figure, a camera passing straight line 351 that is perpendicular to the longer side, closest to the camera 11, of the information display picture 306 and passes through the camera 11 is indicated by a two-dot chain line, a bisecting straight line 352 that divides each of the shorter sides of the information display picture 306 into two equal parts is indicated by a chain line, and trisecting straight lines 353 that divide each of the longer sides of the information display picture 306 into three equal parts are indicated by broken lines. In the information display picture 306, a region that is located on the camera passing straight line 351 and in the vicinity of the camera 11, that is, a top-center region (above the shorter sides bisecting line and between the longer sides trisecting lines) in the information display picture 306, is made a pacer disposition region 365. At least one of horizontally long breathing pacers or indicators 361 and 362 which are moved in the horizontal direction, a vertically long breathing pacer or indicator 363 which is moved in the vertical direction, and a circular breathing pacer or indicator 364 which expands and contracts concentrically is disposed in the pacer disposition region 365. The movement direction of the horizontally long breathing pacers or indicators 361 and 362 crosses the camera passing straight line 351 perpendicularly. The breathing pacers may be located in the pacer disposition region 365; however, it is even preferable that they be located on the camera passing straight line 351.

FIG. 13B shows a state obtained by rotating the monitor 12 and the camera 11 of the apparatus shown in FIG. 13A by 90° counterclockwise. In this case, an information display picture 306 displayed on the monitor 12 is long in the vertical direction (i.e., its longer sides are vertical). In the information display picture 306, a region that is located on the camera passing straight line 351 and in the vicinity of the camera 11, that is, a center-left region (on the left of the shorter sides bisecting line and between the longer sides trisecting lines) in the information display picture 306, is made a pacer disposition region 365. At least one of vertically long breathing pacers or indicators 361a and 362a which are moved in the vertical direction, a horizontally long breathing pacer or indicator 363a which is moved in the horizontal direction, and a circular breathing pacer or indicator 364 which expands and contracts concentrically is disposed in the pacer disposition region 365. The movement direction of the vertically long breathing pacers or indicators 361a and 362a crosses the camera passing straight line 351 perpendicularly. Part of a large GUI like the breathing pacers or indicators 362 and 362a may stick out of the pacer disposition region 365. The breathing pacers may be located in the pacer disposition region 365; however, it is even preferable that they be located on the camera passing straight line 351.

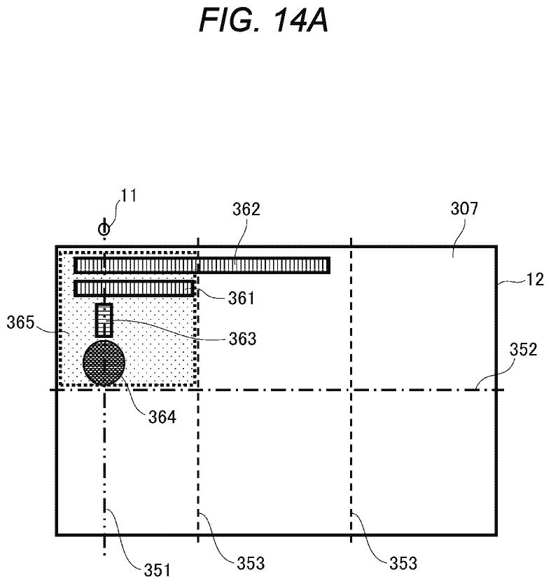

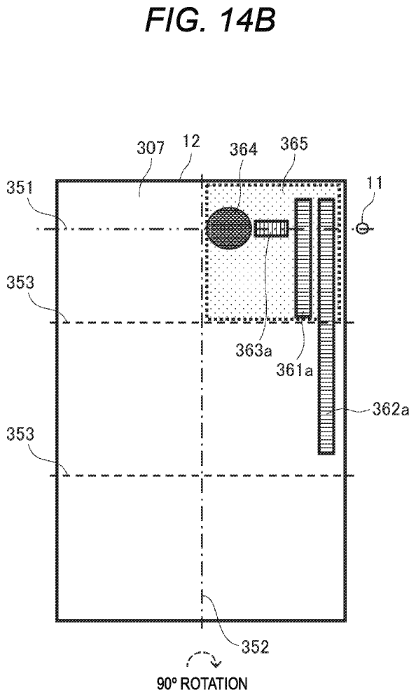

FIGS. 14A and 14B are diagrams showing Arrangement example-2 of GUIs in the information display picture employed in the embodiment. In Arrangement example-2 shown in FIG. 14A, the camera 11 is disposed outside (over) a one end portion (left end portion) of the top longer side of the outer circumference of the display screen of the monitor 12. An information display picture 307 displayed on the monitor 12 is long in the horizontal direction (i.e., its longer sides are horizontal). In the figure, a camera passing straight line 351 that is perpendicular to the longer side, closest to the camera 11, of the information display picture 307 and passes through the camera 11 is indicated by a two-dot chain line, a bisecting straight line 352 that divides each of the shorter sides of the information display picture 307 into two equal parts is indicated by a chain line, and trisecting straight lines 353 that divide each of the longer sides of the information display picture 307 into three equal parts are indicated by broken lines. In the information display picture 307, a region that is located on the camera passing straight line 351 and in the vicinity of the camera 11, that is, a top-left region (above the shorter sides bisecting line and on the left of the left longer sides trisecting line) in the information display picture 307, is made a pacer disposition region 365. At least one of horizontally long breathing pacers or indicators 361 and 362 which are moved in the horizontal direction, a vertically long breathing pacer or indicator 363 which is moved in the vertical direction, and a circular breathing pacer or indicator 364 which expands and contracts concentrically is disposed in the pacer disposition region 365. The breathing pacers may be located in the pacer disposition region 365; however, it is even preferable that they be located on the camera passing straight line 351.

FIG. 14B shows a state obtained by rotating the monitor 12 and the camera 11 of the apparatus shown in FIG. 14A by 90° clockwise. In this case, an information display picture 307 displayed on the monitor 12 is long in the vertical direction (i.e., its longer sides are vertical). In the information display picture 307, a region that is located on the camera passing straight line 351 and in the vicinity of the camera 11, that is, a top-right region (on the right of the shorter sides bisecting line and above the upper longer sides trisecting line) in the information display picture 307, is made a pacer disposition region 365. At least one of vertically long breathing pacers or indicators 361a and 362a which are moved in the vertical direction, a horizontally long breathing pacer or indicator 363a which is moved in the horizontal direction, and a circular breathing pacer or indicator 364 which expands and contracts concentrically is disposed in the pacer disposition region 365. Part of a large GUI like the breathing pacers or indicators 362 and 362a may stick out of the pacer disposition region 365. The breathing pacers may be located in the pacer disposition region 365; however, it is even preferable that they be located on the camera passing straight line 351.

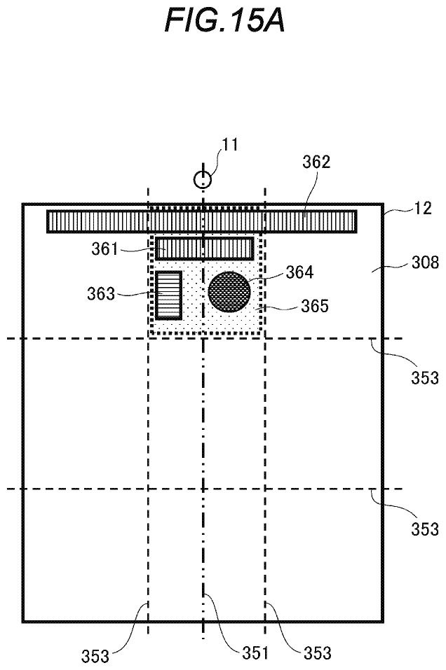

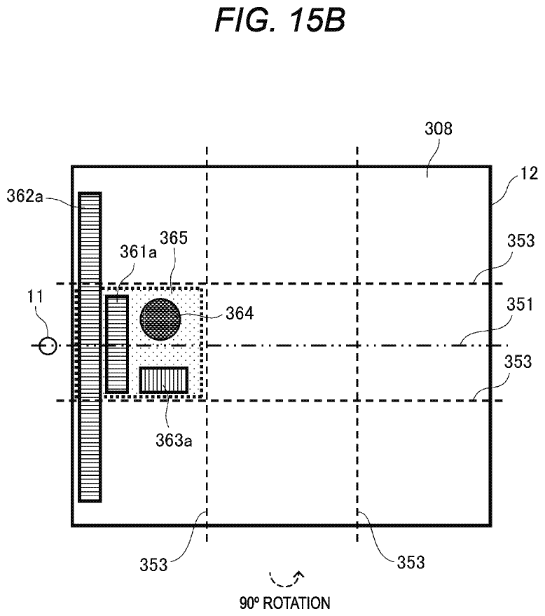

FIGS. 15A and 15B are diagrams showing Arrangement example-3 of GUIs in the information display picture employed in the embodiment. In Arrangement example-3 shown in FIG. 15A, the camera 11 is disposed outside (over) a central portion of the top shorter side of the outer circumference of the display screen of the monitor 12. An information display picture 308 displayed on the monitor 12 is long in the vertical direction (i.e., its longer sides are vertical). In the figure, a camera passing straight line 351 that is perpendicular to the shorter side, closest to the camera 11, of the information display picture 308 and passes through the camera 11 is indicated by a two-dot chain line and trisecting straight lines 353 that divide each of the longer sides or each of the shorter sides of the information display picture 308 into three equal parts are indicated by broken lines. In the information display picture 308, a region that is located on the camera passing straight line 351 and in the vicinity of the camera 11, that is, a top-center region (above the upper longer sides trisecting line and between the shorter sides trisecting lines) in the information display picture 308, is made a pacer disposition region 365. At least one of horizontally long breathing pacers or indicators 361 and 362 which are moved in the horizontal direction, a vertically long breathing pacer or indicator 363 which is moved in the vertical direction, and a circular breathing pacer or indicator 364 which expands and contracts concentrically is disposed in the pacer disposition region 365. The movement direction of the horizontally long breathing pacers or indicators 361 and 362 crosses the camera passing straight line 351 perpendicularly. The breathing pacers may be located in the pacer disposition region 365; however, it is even preferable that they be located on the camera passing straight line 351.

FIG. 15B shows a state obtained by rotating the monitor 12 and the camera 11 of the apparatus shown in FIG. 15A by 90° counterclockwise. In this case, an information display picture 308 displayed on the monitor 12 is long in the horizontal direction (i.e., its longer sides are horizontal). In the information display picture 308, a region that is located on the camera passing straight line 351 and in the vicinity of the camera 11, that is, a center-left region (on the left of the left longer sides trisecting line and between the shorter sides trisecting lines) in the information display picture 308, is made a pacer disposition region 365. At least one of vertically long breathing pacers or indicators 361a and 362a which are moved in the vertical direction, a horizontally long breathing pacer or indicator 363a which is moved in the horizontal direction, and a circular breathing pacer or indicator 364 which expands and contracts concentrically is disposed in the pacer disposition region 365. The movement direction of the vertically long breathing pacers or indicators 361a and 362a crosses the camera passing straight line 351 perpendicularly. Part of a large GUI like the breathing pacers or indicators 362 and 362a may stick out of the pacer disposition region 365. The breathing pacers may be located in the pacer disposition region 365; however, it is even preferable that they be located on the camera passing straight line 351.

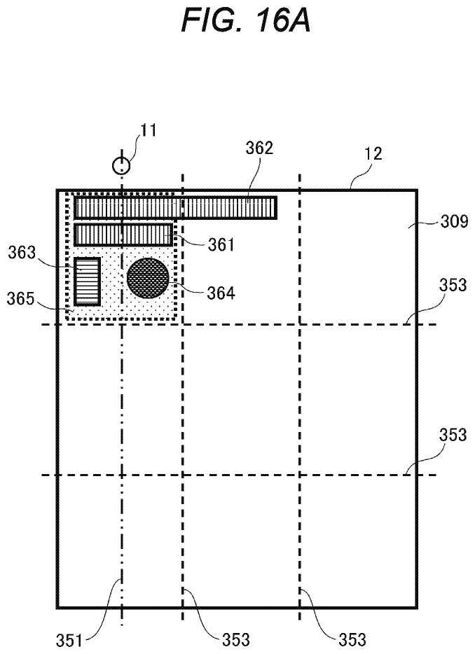

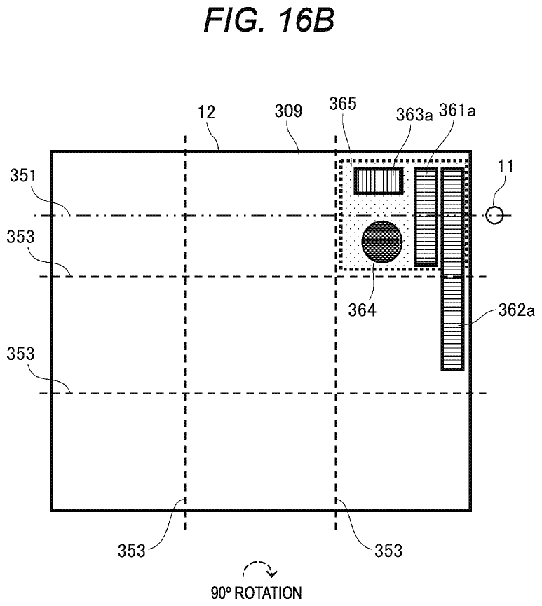

FIGS. 16A and 16B are diagrams showing Arrangement example-4 of GUIs in the information display picture employed in the embodiment. In Arrangement example-4 shown in FIG. 16A, the camera 11 is disposed outside (over) a one end portion (left end portion) of the top shorter side of the outer circumference of the display screen of the monitor 12. An information display picture 309 displayed on the monitor 12 is long in the vertical direction (i.e., its longer sides are vertical). In the figure, a camera passing straight line 351 that is perpendicular to the shorter side, closest to the camera 11, of the information display picture 309 and passes through the camera 11 is indicated by a two-dot chain line and trisecting straight lines 353 that divide each of the longer sides or each of the shorter sides of the information display picture 309 into three equal parts are indicated by broken lines. In the information display picture 309, a region that is located on the camera passing straight line 351 and in the vicinity of the camera 11, that is, a top-left region (above the upper longer sides trisecting line and on the left of the left shorter sides trisecting line) in the information display picture 309, is made a pacer disposition region 365. At least one of horizontally long breathing pacers or indicators 361 and 362 which are moved in the horizontal direction, a vertically long breathing pacer or indicator 363 which is moved in the vertical direction, and a circular breathing pacer or indicator 364 which expands and contracts concentrically is disposed in the pacer disposition region 365. The breathing pacers may be located in the pacer disposition region 365; however, it is even preferable that they be located on the camera passing straight line 351.

FIG. 16B shows a state obtained by rotating the monitor 12 and the camera 11 of the apparatus shown in FIG. 16A by 90° clockwise. In this case, an information display picture 309 displayed on the monitor 12 is long in the horizontal direction (i.e., its longer sides are horizontal). In the information display picture 309, a region that is located on the camera passing straight line 351 and in the vicinity of the camera 11, that is, a top-right region (on the right of the right longer sides trisecting line and above the upper shorter sides trisecting line) in the information display picture 309, is made a pacer disposition region 365. At least one of vertically long breathing pacers or indicators 361a and 362a which are moved in the vertical direction, a horizontally long breathing pacer or indicator 363a which is moved in the horizontal direction, and a circular breathing pacer or indicator 364 which expands and contracts concentrically is disposed in the pacer disposition region 365. Part of a large GUI like the breathing pacers or indicators 362 and 362a may stick out of the pacer disposition region 365. The breathing pacers may be located in the pacer disposition region 365; however, it is even preferable that they be located on the camera passing straight line 351.

As described above, the vital information display apparatus 10 according to the embodiment is equipped with the camera 11 (imaging unit) which images the face of an observation target person (user) and acquires, from an image taken, video data of a detection area including a skin portion of the face of the observation target person; the CPU 13 and the DSP 14 (processing unit) which acquire vital information including information of a human pulse wave from the video data of the detection area and generates an information display picture to be used for performing heart rate variability biofeedback using the vital information; and the monitor 12 (display unit) which displays the information display picture. The camera 11 and the monitor 12 are installed on the same surface in the apparatus body. The processing unit generates, for the heart rate variability biofeedback, an information display picture 30 including a breathing assistant that assists breathing of the observation target person. In the information display picture 30, at least part of the breathing assistant is disposed on a straight line passing through the camera 11 and the straight line passing through the camera 11 crosses a side, closest to the camera 11, of the outer circumference of the monitor 12 perpendicularly. The breathing assistant includes the breathing pacer 31 that instructs the observation target person on a breathing pace. In the information display picture 30, at least part of the breathing pacer 31 is disposed on the straight line passing through the camera 11.

With the above configuration, when heart rate variability biofeedback is performed, the face of an observation target person (user) is shot so as to be located at and with such a imaging position and an angle of view that are suitable for measurement of a heart rate using an image taken by the camera 11 in a state that the observation target person is looking at the breathing pacer 31 which is an example breathing assistant displayed in the information display picture 30. Furthermore, in the information display picture 30, the breathing pacer 31 is disposed in such a range as to allow the user to see it merely by moving his or her line of sight or moving his or her line of sight as little as possible. Thus, an image of the face can always be taken almost squarely by the camera 11 while the user is looking at the breathing pacer 31. As a result, the face of the user can be detected easily and a large image of a skin portion, having a skin color, of the face can be acquired stably. Furthermore, movement of the face of the user can be suppressed and, even if the face is moved, the influence of the face movement can be made small because of a small angular variation with respect to the camera 11. Thus, disorder in the heart rate can be suppressed in acquiring a heart rate from an image taken and vital information relating to a heart rate variation can be acquired stably: the accuracy of detection of vital information can be increased. Furthermore, the usability can be increased by performing heart rate variability biofeedback by detecting vital information without making contact using an image taken.

In the vital information display apparatus 10, the breathing assistant includes the indicator 32 which indicates states of breathing and heartbeats of the observation target person and at least part of the indicator 32 is disposed on the straight line passing through the camera 11 in the information display picture 30. With this measure, in the information display picture 30, the breathing pacer 31 and the indicator 32 are disposed in such a range as to allow the user to see them merely by moving his or her line of sight or moving his or her line of sight as little as possible. Thus, an image of the face can always be taken almost squarely by the camera 11 while the user is looking at the breathing pacer 31 and the indicator 32, whereby the face of the user can be detected easily and a large image of a skin portion of the face can be acquired stably. Thus, disorder in the heart rate can be suppressed in acquiring a heart rate from an image taken and vital information relating to a heart rate variation can be acquired stably: the accuracy of detection of vital information can be increased.

In the vital information display apparatus 10, the breathing assistant includes the indicator 32 which indicates states of breathing and heartbeats of the observation target person. The breathing pacer 31 is to guide a breathing pace of the observation target person in such a manner that at least part of it moves in the information display picture 30, the indicator 32 is to indicate a state of the observation target person in such a manner that at least part of it moves in the information display picture 30, and movement directions of the breathing pacer 31 and the indicator 32 are the same. Various modes are possible such as a mode in which the breathing pacer 31 and the indicator 32 move in the same direction so as to form loci that are straight, for example. The breathing pacer may be given an indicator function by, for example, changing its color so as to be able to also serve as an indicator. This measure allows the user to follow the breathing pacer with his or her eyes easily and to adjust his or her breathing pace and timing so that they match the movement of the breathing pacer. Thus, the heart rate variability biofeedback can be performed properly.

In the vital information display apparatus 10, the breathing assistant includes the indicator 32 which indicates states of breathing and heartbeats of the observation target person. The breathing pacer 31 is to guide a breathing pace of the observation target person in such a manner that at least part of it moves in the information display picture 30, the indicator 32 is to indicate a state of the observation target person in such a manner that at least part of it moves in the information display picture 30, and movement directions of the breathing pacer 31 and the indicator 32 are different from each other. Various modes are possible such as a mode in which the breathing pacer 31 moves so as to form such a locus a mountain-shaped line, a straight line, or a circle and the indicator 32 changes in size and a mode in which the breathing pacer 31 and the indicator 32 move in orthogonal directions. This measure allows the user to adjust his or her breathing pace and timing so that they match the movement of the breathing pacer properly and to recognize the indicator easily. Thus, the heart rate variability biofeedback can be performed properly.

In the vital information display apparatus 10, the breathing assistant includes the indicator 32 which indicates states of breathing and heartbeats of the observation target person. The breathing pacer 31 is to guide a breathing pace of the observation target person in such a manner that at least part of it moves in the information display picture 30, and the indicator 32 is to indicate a state of the observation target person by its state variation. Various modes are possible such as a mode in which the breathing pacer 31 moves so as to form such a locus a mountain-shaped line, a straight line, or a circle and the indicator 32 changes in its state such as color or size. This measure allows the user to adjust his or her breathing pace and timing so that they match the movement of the breathing pacer properly and to recognize the indicator easily. Thus, the heart rate variability biofeedback can be performed properly.

In the vital information display apparatus 10, the movement direction of the breathing pacer 31 crosses the straight line passing through the camera 11 perpendicularly. This measure makes it possible to suppress movement of the face of the user who is looking at the breathing pacer and hence to acquire vital information relating to a heart rate variation stably.

In the vital information display apparatus 10, the breathing pacer 31 is disposed in a pacer disposition region 365 that is located on the straight line passing through the camera 11 in the information display picture 30 and the pacer disposition region 365 is located in a central one of three regions that are obtained by trisecting the information display picture 30 by two straight lines that are parallel with the straight line passing through the camera 11. With this measure, an image of the face can always be taken almost squarely by the camera 11 while the user is looking at the breathing pacer 31. As a result, a large image of a skin portion of the face can be acquired stably, whereby disorder in the heart rate can be suppressed in acquiring a heart rate from an image taken and vital information relating to a heart rate variation can be acquired stably.

In the vital information display apparatus 10, the breathing pacer 31 is disposed in a pacer disposition region 365 that is located on the straight line passing through the camera 11 in the information display picture 30 and the pacer disposition region 365 is located in an end region of three regions that are obtained by trisecting the information display picture 30 by two straight lines that are parallel with the straight line passing through the camera 11. With this measure, an image of the face can always be taken almost squarely by the camera 11 while the user is looking at the breathing pacer 31. As a result, a large image of a skin portion of the face can be acquired stably, whereby disorder in the heart rate can be suppressed in acquiring a heart rate from an image taken and vital information relating to a heart rate variation can be acquired stably.

In the vital information display apparatus 10, the pacer disposition region 365 is located in one, on the side of the camera 11, of two regions that are obtained by bisection by a straight line that is parallel with the straight line passing through the camera 11. With this measure, since the camera 11 and the breathing pacer 31 are disposed close to each other, an image of the face can always be taken almost squarely by the camera 11 while the user is looking at the breathing pacer 31. As a result, vital information relating to a heart rate variation can be acquired stably.

In the vital information display apparatus 10, the movement speed of the breathing pacer 31 changes according to breathing timing. Various modes are possible such as a mode in which among plural intervals obtained by dividing a breathing cycle period the movement speed is set low in a breathing-in start interval, a breathing-in end interval, a breathing-out start interval, and a breathing-out end interval and set higher than the slow speeds in the other intervals and a mode in which the breathing pacer 31 stops, accelerates, moves at a constant speed, decelerates, and stops in this order in each breathing-in interval and each breathing-out interval. With this measure, since the breathing pacer is displayed in such a manner that its movement speed changes according to breathing timing, the user can easily follow the breathing pacer with his or her eyes and adjust his or her breathing pace and timing so that they match the movement of the breathing pacer. Thus, the heart rate variability biofeedback can be performed properly.

In the vital information display 10, the movement speed of the breathing pacer 31 decelerates at least one of a set of a start portion and an end portion of a breathing-in interval and a set of a start portion and an end portion of a breathing-out interval. Alternatively, the breathing pacer 31 makes a constant-speed movement in intervals other than a start portion and an end portion of a breathing-in interval and a start portion and an end portion of a breathing-out interval. With these measures, since the breathing pacer is displayed in such a manner that its movement speed changes according to breathing timing, the user can easily follow the breathing pacer with his or her eyes and adjust his or her breathing pace and timing so that they match the movement of the breathing pacer. Thus, the heart rate variability biofeedback can be performed properly.

The vital information display apparatus 10 is further equipped with the sensor 16 (detection unit) which detects a position of the camera 11, and the processing unit disposes GUIs including the breathing assistant in the information display picture 30 according to the position of the camera 11. With this measure, the face of the user can be shot at and with a imaging position and an angle of view that are suitable for measurement of a heart rate while the user is looking at the breathing pacer 31 which is an example of the breathing assistant and is displayed in the information display picture 30. Thus, a large image of a skin portion of the face can be acquired stably and movement of the user face can be suppressed, whereby vital information relating to a heart rate variation can be acquired stably.

The vital information display apparatus 10 is further equipped with the sensor 16 (detection unit) which detects orientation of the monitor 12, and the processing unit disposes GUIs including the breathing assistant in the information display picture 30 according to the orientation of the monitor 12 and a positional relationship between the camera 11 and the information display picture 30. With this measure, the user face can be shot properly according to orientation of the information display picture 30 and a positional relationship between the camera 11 and the information display picture 30 such as whether a horizontally long or vertical long information display picture 30 is oriented with its major axis extending in the horizontal direction or the vertical direction or orientation of the information display picture 30, rotated, of the vital information display apparatus 10. Thus, a large image of a skin portion of the face can be acquired stably and movement of the user face can be suppressed, whereby vital information relating to a heart rate variation can be acquired stably.

In the vital information display apparatus 10, the processing unit detects rotation of the information display picture 30 on the basis of the orientation of the monitor 12 and changes the GUIs according to orientation of the information display picture 30. With this measure, when the information display picture 30 is rotated, the user face can be shot properly according to orientation of the information display picture 30 and a positional relationship between the information display picture 30 and the camera 11. Thus, a large image of a skin portion of the face can be acquired stably and movement of the user face can be suppressed, whereby vital information relating to a heart rate variation can be acquired stably.

The vital information display method according to the embodiment is a vital information display method employed in the vital information display apparatus 10 which is equipped with the camera 11 (imaging unit) which images the face of an observation target person (user) and the monitor 12 (display unit) which displays vital information acquired from an image taken and in which the monitor 12 is disposed on the same surface as the camera 11 in the body. In this vital information display method, the face of the observation target person is shot by the camera 11, video data of a detection area including a skin portion of the face of the observation target person is acquired from an image taken, vital information including information of a human pulse wave is acquired from video data of the detection area, and an information display picture 30 for performing heart rate variability biofeedback using the vital information is generated In doing so, the information display picture 30 including a breathing assistant that assists breathing of the user is generated for the heart rate variability biofeedback and displayed on the monitor 12. In the information display picture 30, at least part of the breathing assistant is disposed on a straight line passing through the camera 11 and the straight line passing through the camera 11 crosses a side, closest to the camera 11, of the outer circumference of the monitor 12 perpendicularly.

The program according to the embodiment is a program for causing a computer to execute steps of a vital information display method in the vital information display apparatus 10 which is equipped with the camera 11 (imaging unit) which images the face of an observation target person (user) and the monitor 12 which displays vital information acquired from an image taken and in which the monitor 12 is disposed on the same surface as the camera 11 in the body. In the program, the steps include a step of imaging the face of the observation target person by the camera 11 and acquiring, from an image taken, video data of a detection area including a skin portion of the face of the observation target person, and a step of acquiring vital information including information of a human pulse wave from the video data of the detection area and generating an information display picture 30 to be used for performing heart rate variability biofeedback using the vital information. The steps also include a step of generating, for the heart rate variability biofeedback, an information display picture 30 including a breathing assistant that assists breathing of the user, and a step of displaying the information display picture 30 on the monitor 12. In the information display picture 30, at least part of the assistant is disposed on a straight line passing through the camera 11 and the straight line passing through the camera 11 crosses a side, closest to the camera 11, of the outer circumference of the monitor 12 perpendicularly.

Although the various embodiments have been described above with reference to the accompanying drawings, it goes without saying that the invention is not limited to those examples. It is apparent that those skilled in the art could conceive various changes or modifications within the confines of the claims, and they are naturally construed as being included in the technical scope of the invention. And constituent elements of the above-described embodiments may be combined in a desired manner without departing from the spirit and scope of the invention.

The present application is based on Japanese Patent Application No. 2018-023436 filed on Feb. 13, 2018, the disclosure of which is incorporated herein by reference.

### INDUSTRIAL APPLICABILITY

The present disclosure is useful when applied to vital information display apparatus, vital information display methods, and programs capable of increasing the usability when heart rate variability biofeedback is performed.

### DESCRIPTION OF SYMBOLS

10, 104: Vital information display apparatus
11: Camera
12: Monitor
13: CPU
14: DSP
15: Memory
16: Sensor
17: Camera interface
18: Display interface
19: Bus
30, 302, 303, 304, 305, 306, 307, 308, 309: Information display picture
31: Breathing pacer
32: Indicator
33: Face image display
34: Variation component display
35: Vital information display
36: Stop button
37: Display switching button
38: Menu display
39: Acquired data display
351: Camera passing straight line
361, 362, 363, 364: Breathing pacer or indicator
365: Pacer disposition region

1. A vital information display apparatus comprising:
a body;
an imaging unit which images a face of an observation target person and acquires, from an image taken, video data of a detection area including a skin portion of the face of the observation target person;
a processing unit which acquires vital information including information of a human pulse wave from the video data of the detection area and generates an information display picture to be used for performing heart rate variability biofeedback using the vital information; and
a display unit which is installed on the same surface in the body as the imaging unit and displays the information display picture,
wherein the processing unit generates, for the heart rate variability biofeedback, an information display picture including a breathing assistant that assists breathing of the observation target person; and
in the information display picture, at least part of the breathing assistant is disposed on a straight line passing through the imaging unit and the straight line passing through the imaging unit crosses a side, closest to the imaging unit, of an outer circumference of the display unit perpendicularly.

2. The vital information display apparatus according to claim 1,
wherein the breathing assistant includes a breathing pacer which instructs the observation target person on a breathing pace; and
at least a part of the breathing pacer is disposed on the straight line passing through the imaging unit in the information display picture.

3. The vital information display apparatus according to claim 2,
wherein the breathing assistant further includes an indicator which indicates states of breathing and heartbeats of the observation target person; and
at least a part of the indicator is disposed on the straight line passing through the imaging unit in the information display picture.

4. The vital information display apparatus according to claim 2,
wherein the breathing assistant further includes an indicator which indicates states of breathing and heartbeats of the observation target person; and
the breathing pacer guides a breathing pace of the observation target person in such a manner that at least a part of it moves in the information display picture, the indicator indicates a state of the observation target person in such a manner that at least part of it moves in the information display picture, and movement directions of the breathing pacer and the indicator are the same.

5. The vital information display apparatus according to claim 2,
wherein the breathing assistant further includes an indicator which indicates states of breathing and heartbeats of the observation target person; and
the breathing pacer guides a breathing pace of the observation target person in such a manner that at least part of it moves in the information display picture, the indicator indicates a state of the observation target person in such a manner that at least part of it moves in the information display picture, and movement directions of the breathing pacer and the indicator are different from each other.

6. The vital information display apparatus according to claim 2,
wherein the breathing assistant further includes an indicator which indicates states of breathing and heartbeats of the observation target person; and
the breathing pacer guides a breathing pace of the observation target person in such a manner that at least part of it moves in the information display picture, and the indicator indicates a state of the observation target person by its state variation.

7. The vital information display apparatus according claim 4,
wherein the movement direction of the breathing pacer crosses the straight line passing through the imaging unit perpendicularly.

8. The vital information display apparatus according to claim 2,
wherein the breathing pacer is disposed in a pacer disposition region that is located on the straight line passing through the imaging unit in the information display picture, and the pacer disposition region is located in a central one of three regions that are obtained by trisecting the information display picture by two straight lines that are parallel with the straight line passing through the imaging unit.

9. The vital information display apparatus according to claim 2,
wherein the breathing pacer is disposed in a pacer disposition region that is located on the straight line passing through the imaging unit in the information display picture, and the pacer disposition region is located in an end region of three regions that are obtained by trisecting the information display picture by two straight lines that are parallel with the straight line passing through the imaging unit.

10. The vital information display apparatus according to claim 8,
wherein the pacer disposition region is located in one, on the side of the imaging unit, of two regions that are obtained by bisection by a straight line that is parallel with the straight line passing through the imaging unit.

11. The vital information display apparatus according to claim 4,
wherein a movement speed of the breathing pacer changes according to breathing timing.

12. The vital information display apparatus according to claim 11,
wherein the movement speed of the breathing pacer decelerates at least one of a set of a start portion and an end portion of a breathing-in interval and a set of a start portion and an end portion of a breathing-out interval.

13. The vital information display apparatus according to claim 11,
wherein the breathing pacer makes a constant-speed movement in intervals other than a start portion and an end portion of a breathing-in interval and a start portion and an end portion of a breathing-out interval.

14. The vital information display apparatus according to claim 1, further comprising a detection unit which detects a position of the imaging unit,
wherein the processing unit disposes GUIs including the breathing assistant in the information display picture according to the position of the imaging unit.

15. The vital information display apparatus according to claim 1, further comprising a detection unit which detects orientation of the display unit,
wherein the processing unit disposes GUIs including the breathing assistant in the information display picture according to the orientation of the display unit and a positional relationship between the imaging unit and the information display picture.

16. The vital information display apparatus according to claim 15,
wherein the processing unit detects rotation of the information display picture on the basis of the orientation of the display unit and changes the GUIs according to orientation of the information display picture.

17. A vital information display method employed in a vital information display apparatus which comprises a body, a camera which images a face of an observation target person, and a display which displays vital information acquired from an image taken and in which the display is disposed on the same surface as the camera in the body, the vital information display method comprising:
imaging the face of the observation target person by the camera and acquiring, from an image taken, video data of a detection area including a skin portion of the face of the observation target person;
acquiring vital information including information of a human pulse wave from the video data of the detection area, and generating an information display picture to be used for performing heart rate variability biofeedback using the vital information;
during the generating of the information display picture, generating, for the heart rate variability biofeedback, an information display picture including a breathing assistant that assists breathing of the observation target person; and
displaying the information display picture on the display,
wherein in the information display picture, at least a part of the breathing assistant is disposed on a straight line passing through the imaging unit and the straight line passing through the imaging unit crosses a side, closest to the camera, of an outer circumference of the display perpendicularly.

18. A vital information display apparatus comprising:
a memory that stores instructions; and
a processor that, when executing the instructions stored in the memory, performs a process comprising:
imaging, by a camera, a face of an observation target person;
acquiring, from an image taken, video data of a detection area including a skin portion of the face of the observation target person;
acquiring vital information including information of a human pulse wave from the video data of the detection area;
generating an information display picture to be used for performing heart rate variability biofeedback using the vital information; wherein the information display picture includes a breathing assistant that assists breathing of the observation target person; and
displaying, on a display, the information display picture so that at least part of the breathing assistant is disposed on a straight line passing through the camera, wherein the straight line passing through the camera crosses a side, closest to the camera, of an outer circumference of the display perpendicularly.
